(19) United States
(12) Patent Application Publication
(10) Pub. No.: US 2018/0045724 A1
(43) Pub. Date: Feb. 15, 2018

(54) METHODS OF DETECTING INFLAMMATORY MARKERS AND TREATING INFLAMMATORY CONDITIONS IN HUMANS

(71) Applicant: VETICA LABS, INC., San Diego, CA (US)

(72) Inventors: Juan ESTRUCH, San Diego, CA (US); Genevieve HANSEN, San Diego, CA (US)

(21) Appl. No.: 15/674,427

(22) Filed: Aug. 10, 2017

Related U.S. Application Data
(60) Provisional application No. 62/373,310, filed on Aug. 10, 2016.
(60) Provisional application No. 62/417,952, filed on Nov. 4, 2016.

(51) Int. Cl. G01N 33/564; C07K 14/705; C07K 14/47

(52) U.S. Cl. CPC G01N 33/564; C07K 14/4728; C07K 14/70546; G01N 2333/4727; G01N 2333/70546; G01N 2800/065; G01N 2800/062; G01N 2800/7095; G01N 2800/50; C07K 2319/21

(57) ABSTRACT

The present invention provides methods and systems to accurately detect and measure in a biological sample from a patient, endogenous antibodies, e.g., IgA, to inflammatory proteins, which antibodies are useful as diagnostic markers for inflammatory conditions, including bowel disease (IBD), in patients. Such methods and systems identify whether a sample from the patient is associated with an inflammatory condition, by using non-invasive means, thus conveniently providing information useful for guiding treatment decisions.

### REFERENCE TO EARLIER APPLICATIONS

This application claims the benefit of U.S. Provisional Application No. 62/373,310, filed Aug. 10, 2016, and U.S. Provisional Application No. 62/417,952, filed Nov. 4, 2016, the contents of which applications are incorporated by reference herein.

### FIELD OF THE INVENTION

The invention relates generally to the fields of inflammation and immunology, for example inflammatory bowel disease, and more specifically to serological methods and specific algorithms for diagnosing and distinguishing inflammatory conditions, such as inflammatory bowel disease, from other diseases, particularly comprising detecting and measuring endogenous antibodies associated with inflammation, as well as diagnostic kits for carrying out such methods, and methods of treating patients so diagnosed.

### BACKGROUND OF THE INVENTION

Inflammation is usually a normal, healthy response to injury or infection, but sometimes the inflammatory response is disproportionate or abnormal, so that the inflammation, rather than promoting healing, seriously damages normal tissues, resulting in chronic pain, contributing to a wide variety of serious disorders, in some cases increasing the risk of cancer and heart disease, and in some cases even causing death. Inflammatory bowel disease (IBD), for example, is a debilitating and progressive disease involving inflammation of the gastrointestinal tract. Symptoms include abdominal pain, cramping, diarrhea and bleeding.

One indication of such inflammatory diseases is the presence of inflammatory cells such as neutrophils and macrophages at local sites of inflammation. Inflammation is a response of vascularized tissue to infection and/or injury and it is affected by adhesion of leukocytes to the endothelial cells of blood vessels and their infiltration into the surrounding tissues. Such local concentrations can be detected by invasive methods requiring biopsy procedures and pathology analysis. The inflammatory state can also lie systemic, i.e. polypeptides secreted by inflammatory cells become detectable in the blood serum.

Inflammatory bowel disease (IBD) describes idiopathic gastrointestinal disorders characterized by persistent or recurrent gastrointestinal (GI) signs and histological evidence of GI inflammation for which no underlying cause can be found. It includes ulcerative colitis and Crohn's disease, and usually involves severe and chronic diarrhea, pain, fatigue and weight loss. Effective treatment of IBD requires differentiating the condition from other gastrointestinal disorders that do not necessarily involve chronic inflammation. While certain diagnostics have been developed, these diagnostics are not always accurate. The difficulty in diagnosing IBD and differentiating from other superficially similar conditions hampers early and effective treatment.

Currently, diagnosis of IBD typically involves a slow and inefficient process of ruling out other possible causes for signs and symptoms, such as ischemic colitis, infection, irritable bowel syndrome (IBS), diverticulitis, food sensitivities, and colon cancer, and may require expensive endoscopic and advanced imaging procedures.

There have been various efforts to detect and diagnose gastroinflammatory conditions using biomarkers associated with gastrointestinal infection, for example by detecting antibodies to anti-neutrophil cytoplasmic antibody (ANCA), anti-Saccharomyces cerevisiae immunoglobulin A (ASCA-IgA), anti-Saccharomyces cerevisiae immunoglobulin G (ASCA-IgG), an anti-outer membrane protein C (anti-OmpC) antibody, an anti-flagellin antibody, an anti-I2 antibody, and a perinuclear anti-neutrophil cytoplasmic antibody (pANCA), and other biomarkers. See, e.g. US 20060154276A1; WO 2014053996, and US 20100094560A1, all incorporated herein by reference. These markers, however, do not provide a clear means of distinguishing between a transient infection or gastric irritation, and a chronic inflammatory condition such as IBD.

IBD is only one example of a difficult-to-diagnose inflammatory condition. There is a general need for new diagnostic markers for markers that are sensitive and specific for chronic inflammatory diseases, such as IBD, to provide faster, more efficient, less intrusive diagnosis and to distinguish chronic inflammatory diseases from conditions not primarily mediated by inflammation. The present invention addresses these needs and provides related advantages as well.

### BRIEF DESCRIPTION OF THE INVENTION

The present invention provides novel inflammatory markers and methods for detecting them, to aid in diagnosis and monitoring of inflammatory diseases, either on a systemic basis and/or on a localized basis such as in the gastrointestinal tract.

It has surprisingly been discovered that humans, when suffering from inflammatory conditions, produce autoantibodies to proteins such as calprotectin, β-integrins, lactoferritin, and C-reactive protein, which are known to be associated with inflammation. Such autoantibodies have not previously been discovered or characterized. It is unexpected and counter-intuitive that the body would produce antibodies to its own anti-inflammatory proteins, and further that such antibodies could serve as markers for pathological inflammatory conditions such as IBD.

Further validation for this invention is provided in the parallel patent applications by the inventors hereof, describing their work with companion animals. See, WO 2017/079653 and U.S. application Ser. No. 15/592104, each of which applications are incorporated herein by reference in their entirety.

The invention thus provides in one embodiment methods which comprise detecting and/or measuring endogenous immunoglobulin levels to inflammation markers, such as calprotectin and β-integrins, lactoferritin, and/or C-reactive protein, to detect inflammation either on a systemic basis and/or on a localized basis such as in the gastrointestinal tract. These inflammation-associated autoantibodies may be used as markers to identify and characterize inflammatory conditions. In some cases, they may be detected in conjunction with other endogenous antibodies and markers associated with particular inflammatory conditions. For example, in diagnosing IBD, the invention in some embodiments provides for measuring these autoantibodies to inflammation-associated proteins, in conjunction with markers such as endogenous antibodies to polymorphonuclear leukocytes (PMNs) and to microbes found in the gut, as well as markers such as calprotectin, as are known to be associated with IBD.

In certain embodiments, the invention provides novel methods for detecting the presence and/or level of one or more inflammation-associated autoantibodies in a sample obtained from a patient, wherein the inflammation-associated autoantibodies are endogenous antibodies to an inflammatory marker, e.g., selected from one or more of autoantibodies to a calprotectin, an integrin, a lactoferritin, and a C-reactive protein; e.g., wherein the inflammation-associated autoantibodies are IgA antibodies.

In some embodiments, the present invention provides novel methods of detecting inflammation-associated autoantibodies in a patient, for example screening for presence or absence of IBD in patients by detecting specific autoantibodies and classifying whether a sample from a patient is associated with inflammatory bowel disease (IBD) or not. As a non-limiting example, the present invention is useful for classifying a sample from a patient as an IBD sample using empirical data and/or a statistical algorithm. The present invention is also useful for differentiating between IBD subtypes using empirical data and/or a statistical algorithm.

In another aspect, the present invention provides a method for monitoring the progression or regression of an inflammatory condition, e.g., IBD, in patients, the method comprising: (a) determining the presence or level of at least autoantibody to an inflammation-associated proteins, e.g., such as calprotectin, β-integrins, lactoferritin, and C-reactive protein, in a sample from the individual; and (b) determining the presence or severity of IBD in patients using a statistical algorithm based upon the presence or level of the at least autoantibody.

In a related aspect, the present invention provides a method for monitoring drug efficacy in patients receiving drugs useful for treating IBD, the method comprising: (a) determining the presence or level of at least one marker selected from the group consisting of an anti-PMN antibody, antimicrobial antibody, calprotectin and combinations thereof in a sample from the individual; and (b) determining the presence or severity of IBD in the individual using a statistical algorithm based upon the presence or level of the at least one marker.

Thus, in accordance with one aspect of the methods of the present invention, the level of the different markers in a sample from IBD patients is determined and compare to the presence or absence of the same markers in non-IBD patients. The detection of the autoantibodies is performed using immunochemical reagents, and there is a variety of different immunoassay formats in which the methods of the present invention may be performed. Also provided by the present invention are kits for screening patients for inflammatory conditions such as IBD. Suitable kits include immunochemical reagents useful for detecting and determining the level of certain autoantibodies in a sample.

In certain instances, the methods and systems of the present invention compose a step having a “transformation” or “machine” associated therewith. For example, an ELISA technique may be performed to measure the presence or concentration level of many of the markers described herein. An ELISA includes transformation of the marker, e.g., an endogenous-antibody, into a complex between the marker (e.g., the endogenous antibody) and a binding agent (e.g., antigen), which can then be measured with a labeled secondary antibody. In many instances, the label is an enzyme which transforms a substrate into a detectable product. The detectable product measurement can be performed using a plate reader such as a spectrophotometer. In other instances, genetic markers are determined using various amplification techniques such as PCR. Method steps including amplification such as PCR result in the transformation of single or double strands of nucleic acid into multiple strands for detection. The detection can include the use of a fluorophore, which is performed using a machine such as a fluorometer.

Further areas of applicability of the present invention will become apparent from the detailed description provided hereinafter. It should be understood that the detailed description and specific examples, while indicating certain embodiments of the invention, are intended for purposes of illustration only and are not intended to limit the scope of the invention.

### DETAILED DESCRIPTION OF THE INVENTION

The following description of different embodiments is merely exemplary in nature and is in no way intended to limit the invention, its application, or uses.

### Definitions

As used herein, the following terms have the meanings ascribed to them unless specified otherwise.

As used herein, the term “antibody” includes a population of immunoglobulin molecules, which can be polyclonal or monoclonal and of any class and isotype, or a fragment of an immunoglobulin molecule. There are five major classes of immunoglobulins: IgA, IgD, IgE, IgG, and IgM, and several of these may be further divided into subclasses (isotypes), e.g., IgG1, IgG2, IgG3, IgG4, IgA1 (human), IgA2 (human), IgAa (canine), IgAb (canine), IgAc (canine), and IgAd (canine). Such fragment generally comprises the portion of the antibody molecule that specifically binds an antigen. For example, a fragment of an immunoglobulin molecule known in the art as Fab, Fab′ or F(ab′)2 is included within the meaning of the term antibody.

As used herein, the term “endogenous antibodies” refers to antibodies made by or originating from the patient, which can be isolated from the patient's blood or tissue. Typically, endogenous antibodies are generated in response to a foreign antigen, for example in response to a bacterial antigen, as part of the body's natural defense against infection. In certain cases, however, the patient may generate endogenous antibodies against the body's own proteins, such endogenous antibodies being referred to herein as “autoantibodies”. In the context of this application, therefore, endogenous antibodies may refer to autoantibodies to proteins such as calprotectin, β-integrins, lactoferritin, and C-reactive protein, and/or may also include endogenous antibodies to polymorphonuclear leukocytes (PMNs or granulocytes, including neutrophil granulocytes) and/or to microbes found in the gut, or to other antibodies produced by the body which are useful in diagnosing particular conditions. As the patient is a human, the endogenous antibodies would be human antibodies.

The term “endogenous antibodies” is used herein to distinguish from therapeutic or diagnostic antibodies, derived from a source other than the patient, which may for example be administered to the patient or used to detect the presence of antigens in a biological sample (e.g., blood, plasma, urine, tissue, saliva, etc.) from the patient. Therapeutic or diagnostic antibodies would typically be monoclonal antibodies propagated in cell lines, usually derived from antibodies made in other species, e.g., from rodents, or using phage display techniques. Therapeutic antibodies could be complete antibodies or antibody fragments.

“Autoantibody”, as used herein, refers to an endogenous antibody made by the patient against an endogenous antigen, for example against an endogenous protein. The examples herein, for example, describe autoantibodies against endogenous inflammation-related proteins such as calprotectin, integrin, lactoferrin, and/or CRP. Accordingly, where the autoantibody binds to an inflammation-related protein, both the autoantibody and the inflammation-related protein antigen would be from the same individual and the same species, e.g., the autoantibodies generated by the patient are human antibodies, and the endogenous antigen would be a human peptide, e.g., human calprotectin or human integrin. The autoantibody in such a case can be isolated and characterized by its binding to a protein having the same binding epitope as the endogenous antigen.

“Class switching” or “isotype switching” means a change in the phenotype of an immunoglobulin producing cell. Immunoglobulin class switching is a critical step in the generation of the diversified biological effector functions of the antibody response. During the course of an antibody mediated immune response, immunoglobulin producing cells are induced to undergo genetic rearrangements, a process known as class switch recombination (CSR) that results in “switching” of a variable region to different constant region sequence. The identity of the heavy-chain class to which an immunoglobulin-producing cell is switched is believed to be regulated by cytokines. For example, IgA class switching is the process whereby an immunoglobulin-producing cell acquire the expression of IgA, the most abundant antibody isotype in mucosal secretions.

“Inflammation” or “inflammatory condition” as used herein refers to a immunovascular response to a stimuli, for example an immune response to an antigen, a pathogen, or a damaged cell, which is mediated by white blood cells (leukocytes). In some embodiments, the inflammation may be chronic. In some embodiments, the inflammation may be an autoimmune condition, where the immune system causes damage to otherwise normal, non-foreign tissue, as is seen for example in rheumatoid arthritis, multiple sclerosis, and other autoimmune diseases.

The term “inflammatory bowel disease” or “IBD” refers to a chronic inflammation of all or part of the gastrointestinal tract, include, without limitation, the following sub-types: ulcerative colitis, Crohn's disease, lymphoplasmacytic enteritis (LPE), eosinophilic gastroenteritis (EGE) and granulomatous enteritis (GE) Inflammatory bowel diseases are distinguished from all other disorders, syndromes, and abnormalities of the gastroenterological tract, including irritable bowel syndrome (IBS) and transient GI infections, in being characterized by chronic inflammation.

“IBD-associated antibody” refers to an antibody in the serum of the patient to be diagnosed or treated, which is associated with the presence, severity or type of IBD, and so can be considered a marker for IBD. IBD-associated antibodies include for example endogenous antibodies known to be associated with IBD in humans, such as anti-PMN antibodies, anti-yeast antibodies, antimicrobial antibodies, for example antibodies to bacterial OmpC or flagellin proteins, as well as autoantibodies against endogenous inflammation-related proteins such as calprotectin, integrin, lactoferrin, and/or CRP.

The term “sample” includes any biological specimen obtained from a patient. Suitable samples for use in the present invention include, without limitation, whole blood, plasma, serum, saliva, urine, stool, tears, any other bodily fluid, tissue samples (e.g., biopsy), and cellular extracts thereof (e.g., red blood cellular extract). The use of samples such as serum, saliva, and urine is well known in the art (Hashida et al. J. Clin. Lab. Anal., 11:267-286 (1997). One skilled in the art will appreciate that samples such as serum samples can be diluted prior to the analysis of marker levels.

The term “marker” includes any biochemical marker, serological marker, genetic marker, or other clinical or echographic characteristic that can be used to classify a sample from a patient as being associated with an inflammatory condition, such as IBD. Non-limiting examples of markers used herein include anti-PMN antibodies (e.g., APMNA, pAPMNA, cAPMNA, ANSNA, ASAPPA, and the like), antimicrobial antibodies (e.g., anti-Outer-Membrane Protein, anti-OmpC antibodies (ACA), anti-flagellin antibodies (AFA), and the like), lactoferrin, elastase, C-reactive protein (CRP), calprotectin, hemoglobin, and the like and combinations thereof, as well as autoantibodies to endogenous inflammation-related proteins such as calprotectin, integrin, lactoferrin, and/or CRP. The recitation of specific examples of markers associated with inflammatory conditions is not intended to exclude other markers as known in the art and suitable for use in the present invention.

The term “classifying” includes “associating” or “categorizing” a sample or a patient with a disease state or prognosis. In certain instances, “classifying” is based on statistical evidence, empirical evidence, or both. In certain embodiments, the methods and systems of classifying use a so-called training set of samples from patients with known disease states or prognoses. Once established, the training data set serves as a basis, model, or template against which the features of an unknown sample from a patient are compared, in order to classify the unknown disease state or provide a prognosis of the disease state in the patient. In some instances, “classifying” is akin to diagnosing the disease state and/or differentiating the disease state from another disease state. In other instances, “classifying” is akin to providing a prognosis of the disease state in a patient diagnosed with the disease state.

The term “marker profile” includes one, two, three, four, five, six, seven, eight, nine, ten, or more diagnostic and/or prognostic marker(s), wherein the markers can be a serological marker, a protein marker, a genetic marker, and the like. In some embodiments, the marker profile together with a statistical analysis can provide veterinarians valuable diagnostic and prognostic insight. In other embodiments, the marker profile with optionally a statistical analysis provides a projected response to biological therapy. Combining information from multiple diagnostic predictors is often useful, because combining data on multiple markers may provide a more sensitive and discriminating tool for diagnosis or screening applications than any single marker on its own. By using multiple markers (e.g., serological, protein, genetic, etc.) in conjunction with statistical analyses, the assays described herein provide diagnostic, prognostic and therapeutic value by identifying patients with IBD or a clinical subtype thereof, predicting risk of developing complicated disease, assisting in assessing the rate of disease progression (e.g., rate of progression to complicated disease or surgery), and assisting in the selection of therapy.

The term “label,” as used herein, refers to a detectable compound, composition, or solid support, which can be conjugated directly or indirectly (e.g., via covalent or non-covalent means, alone or encapsulated) to a monoclonal antibody or a protein. The label may be detectable by itself (e.g., radioisotope labels, chemiluminescent dye, electrochemical labels, metal chelates, latex particles, or fluorescent labels) or, in the case of an enzymatic label, may catalyze chemical alteration of a substrate compound or composition which is detectable (e.g., enzymes such as horseradish peroxidase, alkaline phosphatase, and the like). The label employed in the current invention could be, but is not limited to alkaline phosphatase; glucose-6-phosphate dehydrogenase (“G6PDH”); horseradish peroxidase (HRP); chemiluminescers such as isoluminol, fluorescers such as fluorescein and rhodamine compounds; ribozymes; and dyes. The label may also be a specific binding molecule which itself may be detectable (e.g., biotin, avidin, streptavidin, digioxigenin, maltose, oligohistidine, e.g., hex-histidine, 2, 4-dinitrobenzene, phenylarsenate, ssDNA, dsDNA, and the like). The utilization of a label produces a signal that may be detected by means such as detection of electromagnetic radiation or direct visualization, and that can optionally be measured.

A monoclonal antibody can be linked to a label using methods well known to those skilled in the art, e.g., Immunochemical Protocols; Methods in Molecular Biology, Vol. 295, edited by R. Bums (2005)). For example, a detectable monoclonal antibody conjugate may be used in any known diagnostic test format like ELISA or a competitive assay format to generate a signal that is related to the presence or amount of an IBD-associated antibody in a test sample.

“Substantial binding” or “substantially binding” refer to an amount of specific binding or recognizing between molecules in an assay mixture under particular assay conditions. In its broadest aspect, substantial binding relates to the difference between a first molecule's incapability of binding or recognizing a second molecule, and the first molecules capability of binding or recognizing a third molecule, such that the difference is sufficient to allow a meaningful assay to be conducted to distinguish specific binding under a particular set of assay conditions, which includes the relative concentrations of the molecules, and the time and temperature of an incubation. In another aspect, one molecule is substantially incapable of binding or recognizing another molecule in a cross-reactivity sense where the first molecule exhibits a reactivity for a second molecule that is less than 25%, e.g. less than 10%, e.g., less than 5% of the reactivity exhibited toward a third molecule under a particular set of assay conditions, which includes the relative concentration and incubation of the molecules. Specific binding can be tested using a number of widely known methods, e.g, an immunohistochemical assay, an enzyme-linked immunosorbent assay (ELISA), a radioimmunoassay (RIA), or a western blot assay.

As used herein, the term “substantially the same amino acid sequence” includes an amino acid sequence that is similar, but not identical to, the naturally-occurring amino acid sequence. For example, an amino acid sequence, i.e., polypeptide, that has substantially the same amino acid sequence as a flagellin protein can have one or more modifications such as amino acid additions, deletions, or substitutions relative to the amino acid sequence of the naturally-occurring flagellin protein, provided that the modified polypeptide retains substantially at least one biological activity of flagellin such as immunoreactivity. The “percentage similarity” between two sequences is a function of the number of positions that contain matching residues or conservative residues shared by the two sequences divided by the number of compared positions times 100. In this regard, conservative residues in a sequence is a residue that is physically or functionally similar to the corresponding reference residue, e.g., that has a similar size, shape, electric charge, chemical properties, including the ability to form covalent or hydrogen bonds, or the like.

“Amino acid consensus sequence,” as used herein, refers to a hypothetical amino acid sequence that can be generated using a matrix of at least two, for example, more than two, aligned amino acid sequences, and allowing for gaps in the alignment, such that it is possible to determine the most frequent amino acid residue at each position. The consensus sequence is that sequence which comprises the amino acids which are most frequently represented at each position. In the event that two or more amino acids are equally represented at a single position, the consensus sequence includes both or all of those amino acids. In some cases, amino acid consensus sequences correspond to a sequence or sub-sequence found in nature. In other cases, amino acid consensus sequences are not found in nature, but represent only theoretical sequences.

“Homology” is an indication that two nucleotide sequences represent the same gene or a gene product thereof, and typically means that that the nucleotide sequence of two or more nucleic acid molecules are partially, substantially or completely identical. When from the same organism, homologous polynucleotides are representative of the same gene having the same chromosomal location, even though there may be individual differences between the polynucleotide sequences (such as polymorphic variants, alleles and the like).

The term “heterologous” refers to any two or more nucleic acid or polypeptide sequences that are not normally found in the same relationship to each other in nature. For instance, a heterologous nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion protein).

As used herein, the term “fragment” includes a peptide, polypeptide or protein segment of amino acids of the full-length protein, provided that the fragment retains reactivity with at least one antibody in sera of disease patients. In some embodiments, the antigen or fragment thereof comprises at the amino-terminus and/or carboxyl-terminus one or more or a combination of tags such as a polyhistidine tag (e.g., 6×His tag), a Small Ubiquitin-like Modifier (SUMO), a glutathione S-transferase (GST), and the like. An “antigenic fragment” is a fragment of a full-length protein that comprises an antibody binding epitope, for example an epitope to which an antibody of interest exhibits substantial binding.

An “epitope” is the antigenic determinant on a polypeptide that is recognized for binding by a paratope on antibodies specific to the polypeptide, for example, an IBD-associated antibody.

Antibodies in the context of the invention may recognize particular epitopes having a sequence of 3 to 11, e.g., 5 to 7, amino acids. The antibody may further be characterized by its binding affinity to the protein, polypeptide or peptide applied in the methods and kits of the invention, and the binding affinity (K_{D}) is, for example, in the nanomolar range, e.g., K_{D} 10⁻⁷ or less, for example, to K_{D} 10⁻⁹ to 10⁻¹⁰. Particular antibodies used in the invention are the autoantibodies as described, as well as IBD-associated antibodies found in the serum of patients with IBD, and monoclonal or polyclonal antibodies directed against antibodies, used as detection antibodies.

The term “clinical factor” includes a symptom in a patient that is associated with IBD. Examples of clinical factors include, without limitation, diarrhea, abdominal pain and/or discomfort, cramping, fever, anemia, hypoproteinemia, weight loss, anxiety, lethargy, and combinations thereof. In some embodiments, a diagnosis of IBD is based upon a combination of analyzing the presence or level of one or more markers in a patient using statistical algorithms and determining whether the patient has one or more clinical factors.

The term “prognosis” includes a prediction of the probable course and outcome of IBD or the likelihood of recovery from the disease. In some embodiments, the use of statistical algorithms provides a prognosis of IBD in a patient. For example, the prognosis can be surgery, development of a clinical subtype of IBD, development of one or more clinical factors, development of intestinal cancer, or recovery from the disease.

The term “prognostic profile” includes one, two, three, four, five, six, seven, eight, nine, ten, or more marker(s) of a patient, wherein the marker(s) can be a serological marker, a protein marker, a genetic marker, and the like. A statistical analysis transforms the marker profile into a prognostic profile. An example of statistical analysis can be defined, but not limited to, analysis by quartile scores and the quartile score for each of the markers can be summed to generate a quartile sum score.

The term “diagnosing IBD” includes the use of the methods, systems, and code of the present invention to determine the presence or absence of IBD in a patient. The term also includes methods, systems, and code for assessing the level of disease activity in a patient. The term “monitoring the progression or regression of IBD” includes the use of the methods, systems, and code of the present invention to determine the disease state (e.g., presence or severity of IBD) of a patient. In certain instances, the results of a statistical algorithm are compared to those results obtained for the same patient at an earlier time. In some aspects, the methods, systems, and code of the present invention can also be used to predict the progression of IBD, e.g., by determining a likelihood for IBD to progress either rapidly or slowly in a patient based on the presence or level of at least one marker in a sample. In other aspects, the methods, systems, and code of the present invention can also be used to predict the regression of IBD, e.g., by determining a likelihood for IBD to regress either rapidly or slowly in a patient based on the presence or level of at least one marker in a sample.

The term “diagnosing an inflammatory condition” includes the use of the methods, systems, and code of the present invention to determine the presence or absence of an inflammatory condition in a patient which is a human or nonhuman mammal. The term also includes methods, systems, and code for assessing the level of disease activity in the patient. The term “monitoring the progression or regression of inflammation” includes the use of the methods, systems, and code of the present invention to determine the disease state (e.g., presence or severity of inflammation) of the patient. In certain instances, the results of a statistical algorithm are compared to those results obtained for the same patient at an earlier time. In some aspects, the methods, systems, and code of the present invention can also be used to predict the progression of inflammation, e.g., by determining a likelihood for the inflammation to progress either rapidly or slowly in the patient based on the presence or level of at least one marker in a sample. In other aspects, the methods, systems, and code of the present invention can also be used to predict the regression of inflammation, e.g., by determining a likelihood for inflammation to regress either rapidly or slowly in the patient based on the presence or level of at least one marker in a sample.

As used herein, the term “sensitivity” refers to the probability that a diagnostic method, system, or code of the present invention gives a positive result when the sample is positive, e.g., having IBD or a clinical subtype thereof. Sensitivity is calculated as the number of true positive results divided by the sum of the true positives and false negatives. Sensitivity essentially is a measure of how well a method, system, or code of the present invention correctly identifies those with IBD or a clinical subtype thereof from those without the disease. The statistical algorithms can be selected such that the sensitivity of classifying IBD or a clinical subtype thereof is at least about 60%, and can be, for example, at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “specificity” refers to the probability that a diagnostic method, system, or code of the present invention gives a negative result when the sample is not positive, e.g., not having IBD or a clinical subtype thereof. Specificity is calculated as the number of true negative results divided by the sum of the true negatives and false positives. Specificity essentially is a measure of how well a method, system, or code of the present invention excludes those who do not have IBD or a clinical subtype thereof from those who have the disease. The statistical algorithms can be selected such that the specificity of classifying IBD or a clinical subtype thereof is at least about 50%, for example, at least about 50%, 55%, 60%, 65%, 70%, 75%, 80%, 85%, 86%, 87%, 88%, 89%, 90%, 91%, 92%, 93%, 94%, 95%, 96%, 97%, 98%, or 99%.

As used herein, the term “negative predictive value” or “NPV” refers to the probability that an individual identified as not having IBD or a clinical subtype thereof actually does not have the disease. Negative predictive value can be calculated as the number of true negatives divided by the sum of the true negatives and false negatives. Negative predictive value is determined by the characteristics of the diagnostic method, system, or code as well as the prevalence of the disease in the patient population analyzed. The statistical algorithms can be selected such that the negative predictive value in a population having a disease prevalence is in the range of about 50% to about 99% and can be, for example, at least about 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “positive predictive value” or “PPV” refers to the probability that an individual identified as having IBD or a clinical subtype thereof actually has the disease. Positive predictive value can be calculated as the number of true positives divided by the sum of the true positives and false positives. Positive predictive value is determined by the characteristics of the diagnostic method, system, or code as well as the prevalence of the disease in the patient population analyzed. The statistical algorithms can be selected such that the positive predictive value in a population having a disease prevalence is in the range of about 70% to about 99% and can be, for example, at least about 70%, 75%, 80%, 85%, 86%, 87%, 88%, 89%, 90%, 91%, 92%, 93%, 94%, 95%, 96%, 97%, 98%, or 99%.

Predictive values, including negative and positive predictive values, are influenced by the prevalence of the disease in the patient population analyzed. In the methods, systems, and code of the present invention, the statistical algorithms can be selected to produce a desired clinical parameter for a clinical population with a particular IBD prevalence. For example, learning statistical classifier systems can be selected for an IBD prevalence of up to about 1%, 2%, 3%, 4%, 5%, 6%, 7%, 8%, 9%, 10%, 15%, 20%, 25%, 30%, 35%, 40%, 45%, 50%, 55%, 60%, 65%, or 70%, which can be seen, e.g., in a veterinarian office.

As used herein, the term “overall agreement” or “overall accuracy” refers to the accuracy with which a method, system, or code of the present invention classifies a disease state. Overall accuracy is calculated as the sum of the true positives and true negatives divided by the total number of sample results and is affected by the prevalence of the disease in the patient population analyzed. For example, the statistical algorithms can be selected such that the overall accuracy in a patient population having a disease prevalence is at least about 60%, and can be, for example, at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%.

The term “correlating” as used herein in reference to the use of biomarkers refers to comparing the presence or amount of the biomarker(s) in a patient to its presence or amount in patients known to suffer from, or known to be at risk of, a given condition; or in patients known to be free of a given condition. Often, this takes the form of comparing an assay result in the form of a biomarker concentration to a predetermined threshold selected to be indicative of the occurrence or nonoccurrence of a disease or the likelihood of some future outcome.

Population studies may also be used to select a decision threshold using Receiver Operating Characteristic (“ROC”) analysis to distinguish a diseased subpopulation from a nondiseased subpopulation. A false positive in this case occurs when the sample tests positive, but actually does not have the disease. A false negative, on the other hand, occurs when the sample tests negative, suggesting they are healthy, when they actually do have the disease. To draw a ROC curve, the true positive rate (TPR) and false positive rate (FPR) are determined. Since TPR is equivalent with sensitivity and FPR is equal to 1-specificity, the ROC graph is sometimes called the sensitivity vs (1-specificity) plot. A perfect test will have an area under the ROC curve of 1.0; a random test will have an area of 0.5. A threshold is selected to provide an acceptable level of specificity and sensitivity.

These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained it two groups considered if the groups are of continuous data, or to the Wilcoxon test of ranks.

The term “statistical algorithm” or “statistical process” includes any of a variety of statistical analyses used to determine relationships between variables. In the present invention, the variables are the presence or level of at least one marker of interest. Any number of markers can be analyzed using a statistical algorithm described herein. For example, the presence or levels of 1, 2, 3, 4, 5, 6, 7, 8, 9, 10, 11, 12, 13, 14, 15, 16, 17, 18, 19, 20, 25, 30, 35, 40, 45, 50, or more markers can be included in a statistical algorithm. In one embodiment, logistic regression is used. In another embodiment, linear regression is used. In certain instances, the statistical algorithms of the present invention can use a quantile measurement of a particular marker within a given population as a variable. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present invention can also include the use of percentile ranges of marker levels (e.g., textiles, quartile, quintiles, etc.), or their cumulative indices (e.g., quartile sums of marker levels, etc.) as variables in the algorithms (just as with continuous variables).

The statistical algorithms of the present invention comprise one or more learning statistical classifier systems. As used herein, the term “learning statistical classifier system” includes a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used. Examples of learning statistical classifier systems include, but are not limited to, those using inductive learning (e.g., decision/classification trees such as random forests, classification and regression trees (C&RT), boosted trees, etc.), and genetic algorithms and evolutionary programming.

The learning statistical classifier systems described herein can be trained and tested using a cohort of samples (e.g., serological samples) from healthy and IBD patients. For example, samples from patients diagnosed by a veterinarian as having IBD using a biopsy and/or endoscopy are suitable for use in training and testing the learning statistical classifier systems of the present invention. Samples from healthy patients can include those that were not identified as IBD samples. One skilled in the art will know of additional techniques and diagnostic criteria for obtaining a cohort of patient samples that can be used in training and testing the learning statistical classifier systems of the present invention.

The term “optimizing therapy in a patient having IBD” includes the use of methods, systems, and code of the present invention to determine the course of therapy for a patient before a therapeutic agent (e.g., IBD drug) has been administered. In certain instances, the results of a statistical algorithm are compared to those results obtained for the same patient at an earlier time during the course of therapy. As such, a comparison of the results provides an indication for the need to change the course of therapy or an indication for the need to increase or decrease the dose of the current course of therapy. The term “course of therapy” includes any therapeutic approach taken to relieve or prevent one or more symptoms (i.e., clinical factors) associated with IBD. The term encompasses administering any compound, drug, procedure, or regimen useful for improving the health of a patient with IBD and includes any of the therapeutic agents (e.g., IBD drugs) described above as well as surgery.

The term “therapeutically effective amount or dose” includes a dose of a drug that is capable of achieving a therapeutic effect in a patient in need thereof. For example, a therapeutically effective amount of a drug useful for treating IBD can be the amount that is capable of preventing or relieving one or more symptoms associated with IBD. The exact amount can be ascertainable by one skilled in the art using known techniques broadly reported in Pharmaceutical dosage and compounding books.

The term “therapeutic profile” includes one, two, three, four, five, six, seven, eight, nine, ten, or more marker(s) of an individual, wherein the marker(s) can be a serological marker, a protein marker, a genetic marker, and the like. A statistical analysis transforms the marker profile into a therapeutic profile. An example of statistical analysis can be defined, but not limited to, by quartile scores and the quartile score for each of the markers can be summed to generate a quartile sum score.

The term “efficacy profile” includes one, two, three, four, five, six, seven, eight, nine, ten, or more marker(s) of an individual, wherein the markers can be a serological marker, a protein marker, a genetic marker, and the like, and wherein each of the markers changes with therapeutic administration. In certain instances, the marker profile is compared to the efficacy profile in order to assess therapeutic efficacy. In certain aspects, the efficacy profile is equivalent to the marker profile, but wherein the markers are measured later in time. In certain other aspects, the efficacy profile corresponds to a marker profile from inflammation patients, including IBD patients who responded to a particular therapeutic agent or drug. In these aspects, similarities or differences between the test marker profile and the reference efficacy profile indicate whether that particular drug is suitable or unsuitable for the treatment of inflammation, e.g., IBD.

In certain instances, the methods of the invention are used in order to prognosticate the progression of IBD. The methods can be used to monitor the disease, both progression and regression. The term “monitoring the progression or regression of IBD” includes the use of the methods and marker profiles to determine the disease state (e.g., presence or severity of IBD) of a patient. In certain instances, the results of a statistical analysis are compared to those results obtained for the same patient at an earlier time. In some aspects, the methods, systems, and code of the present invention can also be used to predict the progression of IBD, e.g., by determining a likelihood for IBD to progress either rapidly or slowly in a patient based on the presence or level of at least one marker in a sample. In other aspects, the methods, systems, and code of the present invention can also be used to predict the regression of IBD, e.g., by determining a likelihood for IBD to regress either rapidly or slowly in an individual based on the presence or level of at least one marker in a sample.

In certain instances, the methods of the invention are used in order to prognosticate the progression of an inflammatory condition. The methods can be used to monitor the disease, both progression and regression. The term “monitoring the progression or regression of inflammation” includes the use of the methods and marker profiles to determine the disease state (e.g., presence or severity of inflammation) of a patient, which may be a human or a nonhuman mammal. In certain instances, the results of a statistical analysis are compared to those results obtained for the same patient at an earlier time. In some aspects, the methods, systems, and code of the present invention can also be used to predict the progression of inflammation, e.g., by determining a likelihood for inflammation to progress either rapidly or slowly in the patient based on the presence or level of at least one marker in a sample. In other aspects, the methods, systems, and code of the present invention can also be used to predict the regression of IBD, e.g., by determining a likelihood for inflammation to regress either rapidly or slowly in the patient based on the presence or level of at least one marker in a sample.

The term “monitoring drug efficacy in a patient receiving a drug useful for treating IBD” includes the determination of a marker profile, alone or in combination with the application of a statistical analysis, to determine the disease state (e.g., presence or severity of IBD) of a patient after a therapeutic agent for treating IBD has been administered.

### II. Autoantibodies as Markers for Inflammatory Conditions

Inflammation is a crucial process in the normal defense mechanisms against various pathogens, and leukocytes are the principal cellular mediators of inflammation. Inflammation is characterized histologically by the accumulation of leukocytes in the affected tissue due to migration of circulating leukocytes out of the vasculature, a process which is actively mediated and precisely controlled by leukocytes, the cytokines they produce, and the vascular endothelium. However, excessive or uncontrolled inflammatory responses can lead to the pathologic inflammation seen in many rheumatologic and inflammatory disorders.

Calprotectin and integrins are two classes of proteins that are intimately related to these physiological processes, with their expression, activation and accumulation being tightly controlled under normal conditions. Dysregulation of these proteins have been associated with specific disease conditions like dysregulation of α4β1, α4β7, and αEβ7 integrins may all play a contributory role in the progression of chronic forms of demyelinating disease leading to some forms of multiple sclerosis; dysregulation of α1β2 associated with psoriasis; and α4-type integrins being associated with celiac and other skin-related, gluten-sensitivity diseases.

Calprotectin has commonly been used as a marker to distinguish between organic and functional gastrointestinal disease and for the early diagnosis of inflammatory bowel disease. Calprotectin is a 24 kDa dimer of calcium binding polypeptides S100A8 and S100A9. The complex accounts for up to 60% of the soluble polypeptide content of the neutrophil cytosol and is resistant to enzymatic degradation, and can be measured in feces. A number of assays for calprotectin detection and quantification are already known and generally used to determine calprotectin levels in different body fluids and feces. S100 polypeptides, specially calprotectin and S100Al2 have been studied extensively in human IBD populations and their serum and mucosal levels have been shown to be elevated with IBD. Some studies on calprotectin levels in serum and feces have also been performed in non-human animals and similar trends have been reported, albeit they are very limited.

All estimations of calprotectin in the different body fluids have been done by direct measurement of the polypeptide in different formats but mostly based on the use of antibodies against calprotectin itself, wherein the antibodies are typically monoclonal antibodies, usually murine, made for the purpose of detecting and measuring calprotectin.

Endogenous antibodies to calprotectin, as described herein, have not been described, or associated with inflammatory conditions. The present invention includes methods that determine and quantify endogenous immunoglobulin levels to calprotectin and its complexes in defined cohorts and associating those levels to defined clinical profiles.

Integrins are heterodimeric cell surface receptors which enable adhesion, proliferation, and migration of cells by recognizing binding motifs in extracellular matrix (ECM) polypeptides. As transmembrane linkers between the cytoskeleton and the ECM, they are able to recruit a huge variety of polypeptides and to influence cell processes. Integrins mediate cell-to-cell interactions and are critical homing mechanisms for many biological processes. Alpha-4 integrin is expressed by circulating leukocytes and forms heterodimeric receptors in conjunction with either the beta-1 or the beta-7 integrin subunit. Both alpha-4 beta-1 (α4β1, or very late antigen-4 (VLA-4)) and alpha-4 beta-7 (α4β7) dimers play a role in the migration of leukocytes across the vascular endothelium and contribute to cell activation and survival within the parenchyma. The α4β7 integrin, known as the gut mucosal homing receptor, acts as a homing receptor that mediates lymphocyte migration from gut inductive sites were the immune responses are first induced to the lamina propria.

Integrin-mediated interactions with the extracellular matrix (ECM) are required for the attachment, cytoskeletal organization, mechanosensing, migration, proliferation, differentiation and survival of cells in the context of a multitude of biological processes including fertilization, implantation and embryonic development, immune response, bone resorption and platelet aggregation. Integrins also function in pathological processes such as inflammation, wound healing, angiogenesis, and tumor metastasis.

Many integrins are circulating receptors that are constantly redistributed, internalized and turned over. Because of this, their direct quantification as target antigens is very challenging and has limited its direct measurement to be associated with any clinical conditions.

Endogenous antibodies to integrins as described herein have not been previously described, nor are they known to be associated with inflammatory conditions. The present invention includes methods that enable the quantification of endogenous immunoglobulin levels to integrins by measuring the titers of antibodies specifically recognizing the integrin, and associating them to defined clinical profiles.

Lactoferrin is a protein originally isolated from milk but later found to be present in various other secretory fluids such as saliva, tears and mucosal secretions, and in the granules of neutrophils. Lactoferrin is a potent antimicrobial agent. By sequestering free iron, it can starve bacteria of this essential nutrient. It also binds to bacterial LPS and bacterial cell surface proteins, interfering with bacterial adhesion and disrupting bacterial cell walls or membranes. In inflammatory conditions, plasma levels of lactoferrin may be substantially elevated due to the release of lactoferrin from neutrophil granules.

Endogenous antibodies to lactoferrins as described herein have not been previously described, nor are they known to be associated with inflammatory conditions. The present invention includes methods that enable the quantification of endogenous immunoglobulin levels to lactoferrins by measuring the titers of antibodies specifically recognizing the lactoferrin, and associating them to defined clinical profiles.

C-reactive protein (CRP) is a pentameric protein released by the liver in response to IL-6 released by macrophages and T cells. It binds to the phosphocholine expressed on the surface of dead or dying cells, including some bacteria, and activates the complement system, promoting phagocytosis by macrophages, which clears necrotic and apoptotic cells and bacteria. CRP levels rise rapidly and dramatically in response to inflammation, so it is a good marker for inflammation, and various techniques have been developed to measure CRP levels in order to diagnose and monitor inflammation.

Endogenous antibodies to CRP as described herein have not been previously described, or associated with inflammatory conditions. The present invention includes methods that enable the quantification of endogenous immunoglobulin levels to CRP by measuring the titers of antibodies specifically recognizing the CRP, and associating them to defined clinical profiles.

In each case, the correlation between inflammation and the presence and level of autoantibodies to the foregoing inflammatory markers is particularly marked for IgA autoantibodies to the inflammatory markers.

In certain embodiments, endogenous antibodies to other antigens are also detected, and may be useful to help further characterize the patient's condition. For example, the method may additionally comprise detecting the presence and/or level of one or more endogenous antibodies associated with food sensitivity in a sample (e.g., a sample is selected from one or more of whole blood, serum, plasma, stool, and intestinal tissue) obtained from the patient, wherein the endogenous antibodies are selected from one or more of endogenous antibodies to one or more proteins associated with food sensitivity, e.g., selected from one or more of gliadin, zein, amylase inhibitor, or tissue transglutaminase (e.g. TTG2, or TG3), and/or detecting the presence and/or level of one or more endogenous antibodies associated with gastrointestinal infection, including endogenous antibodies to polymorphonuclear leukocytes (PMNs or granulocytes, including neutrophil granulocytes) and/or endogenous antibodies to microbes found in the gut.

### III. Assays

Any of a variety of assays, techniques, and kits known in the art can be used to determine the presence or level of one or more markers in a sample to classify whether the sample is associated with IBD or a clinical subtype thereof.

The present invention relies, in part, on determining the presence or level of at least one marker in a sample obtained from a patient. As used herein, the term “determining the presence of at least one marker” includes determining the presence of each marker of interest by using any quantitative or qualitative assay known to one of skill in the art. In certain instances, qualitative assays that determine the presence or absence of a particular trait, variable, or biochemical or serological substance (e.g., protein or antibody) are suitable for detecting each marker of interest. In certain other instances, quantitative assays that determine the presence or absence of RNA, protein, antibody, or activity are suitable for detecting each marker of interest. As used herein, the term “determining the level of at least one marker” includes determining the level of each marker of interest by using any direct or indirect quantitative assay known to one of skill in the art. In certain instances, quantitative assays that determine, for example, the relative or absolute amount of RNA, protein, antibody, or activity are suitable for determining the level of each marker of interest. One skilled in the art will appreciate that any assay useful for determining the level of a marker is also useful for determining the presence or absence of the marker.

Flow cytometry can be used to determine the presence or level of one or more markers in a sample. Such flow cytometry assays, including bead based immunoassays (see, e.g. Nolan, J. P. and Mandy, F. Cytometry 69:318-325 (2006).

Phage display technology for expressing a recombinant antigen specific for a marker can also be used to determine the presence or level of one or more markers in a sample. Phage particles expressing an antigen specific for, e.g., an antibody marker can be anchored, if desired, to a multi-well plate using an antibody such as an anti-phage monoclonal antibody (Felici et al, “Phage-Displayed Peptides as Tools for Characterization of Human Sera” in Abelson (Ed.), Methods Enzymol. 267:116-129 (1996).

A variety of immunoassay techniques, including competitive and non-competitive immunoassays (e.g., The immunoassay handbook 4^{th} edition, David Wild ed. Newnes, 2013) can be used to determine the presence or level of one or more markers in a sample. The term immunoassay encompasses techniques including, without limitation, enzyme immunoassays (EIA) such as enzyme multiplied immunoassay technique (EMIT), enzyme-linked immunosorbent assay (ELISA), direct ELISA, antigen capture ELISA, sandwich ELISA, IgM antibody capture ELISA (MAC ELISA), and microparticle enzyme immunoassay (META); capillary electrophoresis immunoassays (CEIA); radioimmunoassays (RIA); immunoradiometric assays (IRMA); fluorescence polarization immunoassays (FPIA); and chemiluminescence assays (CL). Liposome immunoassays, such as flow-injection liposome immunoassays and liposome immunosensors, are also suitable for use in the present invention. In addition, nephelometry assays, in which the formation of protein/antibody complexes results in increased light scatter that is converted to a peak rate signal as a function of the marker concentration, are suitable for use in the present invention. Nephelometry assays are commercially available from Beckman Coulter (Brea, C A; Kit #449430) and can be performed using a Behring Nephelometer Analyzer.

Antigen capture ELISA can be useful for determining the presence or level of one or more markers in a sample. For example, in an antigen capture ELISA, an antibody directed to a marker of interest is bound to a solid phase and sample is added such that the marker is bound by the antibody. After unbound proteins are removed by washing, the amount of bound marker can be quantitated using, e.g., a radioimmunoassay (see, e.g., Harlow and Lane, Antibodies: A Laboratory Manual, Cold Spring Harbor Laboratory, New York, 1988)). Sandwich ELISA can also be suitable for use in the present invention. For example, in a two-antibody sandwich assay, a first antibody is bound to a solid support, and the marker of interest is allowed to bind to the first antibody. The amount of the marker is quantitated by measuring the amount of a second antibody that binds the marker. The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

A radioimmunoassay using, for example, an iodine-125 (¹²⁵I) labeled secondary antibody (Harlow and Lane, supra) is also suitable for determining the presence or level of one or more markers in a sample. A secondary antibody labeled with a chemiluminescent marker can also be suitable for use in the present invention. A chemiluminescence assay using a chemiluminescent secondary antibody is suitable for sensitive, non-radioactive detection of marker levels. Such secondary antibodies can be obtained commercially from various sources, e.g., Amersham Lifesciences, Inc. (Arlington Heights, Ill.).

The immunoassays described above are particularly useful for determining the presence or level of one or more markers in a sample. As a non-limiting example, a fixed PMN ELISA is useful for determining whether a patient sample is positive for APMNA or for determining APMNA levels. Similarly, an ELISA using yeast cell wall phosphopeptidomannan is useful for determining whether a patient sample is positive for AYA-IgA, AYA-IgG, and/or AYA-IgM, or for determining AYA-IgA, AYA-IgG, and/or AYA-IgM levels. An ELISA using OmpC protein or a fragment thereof is useful for determining whether a patient sample is positive for anti-OmpC antibodies, or for determining anti-OmpC antibody levels. An ELISA using flagellin protein or a fragment thereof is useful for determining whether a patient sample is positive for anti-flagellin antibodies, or for determining anti-flagellin antibody levels. An ELISA using calprotectin or a fragment thereof is useful for determining whether a patient sample is positive for calprotectin antibodies, or for determining calprotectin antibody levels. In addition, the immunoassays described above are particularly useful for determining the presence or level of other markers in a patient sample.

Specific immunological binding of the antibody to the marker of interest can be detected directly or indirectly. Direct labels include fluorescent or luminescent tags, metals, dyes, radionuclides, and the like, attached to the antibody. An antibody labeled with iodine-125 (¹²⁵I) can be used for determining the levels of one or more markers in a sample. A chemiluminescence assay using a chemiluminescent antibody specific for the marker is suitable for sensitive, non-radioactive detection of marker levels. An antibody labeled with fluorochrome is also suitable for determining the levels of one or more markers in a sample. Examples of fluorochromes include, without limitation, DAPI, fluorescein, Hoechst 33258, R-phycocyanin, B-phycoerythrin, R-phycoerythrin, rhodamine, Texas red, and lissamine. Secondary antibodies linked to fluorochromes can be obtained commercially, e.g., goat F(ab′)2 anti-human IgG-FITC is available from Tago Immunologicals (Burlingame, Calif.).

Indirect labels include various enzymes well-known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.). A useful secondary antibody linked to an enzyme can be obtained from a number of commercial sources, e.g., goat anti-dog IgG-alkaline phosphatase can be purchased from Jackson ImmunoResearch (West Grove, Pa.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis of the amount of marker levels can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

Quantitative western blotting can also be used to detect or determine the presence or level of one or more markers in a sample. Western blots can be quantitated by well-known methods such as scanning densitometry or phosphorimaging. As a non-limiting example, protein samples are electrophoresed on 10% SDS-PAGE Laemmli gels. Primary murine monoclonal antibodies are reacted with the blot, and antibody binding can be confirmed to be linear using a preliminary slot blot experiment. Goat anti-mouse horseradish peroxidase-coupled antibodies (BioRad) are used as the secondary antibody, and signal detection performed using chemiluminescence, for example, with the Renaissance chemiluminescence kit (New England Nuclear; Boston, Mass.) according to the manufacturer's instructions. Autoradio graphs of the blots are analyzed using a scanning densitometer (Molecular Dynamics; Sunnyvale, Calif.) and normalized to a positive control. Values are reported, for example, as a ratio between the actual value to the positive control (densitometric index). Such methods are well known in the art.

Alternatively, a variety of immunohistochemical assay techniques can be used to determine the presence or level of one or more markers in a sample. The term “immunohistochemical assay” encompasses techniques that utilize the visual detection of fluorescent dyes or enzymes coupled (i.e., conjugated) to antibodies that react with the marker of interest using fluorescent microscopy or light microscopy and includes, without limitation, direct fluorescent antibody assay, indirect fluorescent antibody (WA) assay, anticomplement immunofluorescence, avidin-biotin immunofluorescence, and immunoperoxidase assays. An IFA assay, for example, is useful for determining whether a patient sample is positive for APMNA, the level of APMNA, whether a patient sample is positive for p APMNA, the level of pAPMNA, and/or an APMNA staining pattern (e.g., cAPMNA, pAPMNA, NSNA, and/or SAPPA staining pattern). The concentration of APMNA in a sample can be quantitated, e.g., through endpoint titration or through measuring the visual intensity of fluorescence compared to a known reference standard.

In another embodiment, the detection of antibodies may utilize Agglutination-PCR (ADAP), e.g., as described in Tsai, et al. ACS Cent. Sci., 2016, 2 (3), pp 139-147, e.g., using a qPCR assay to ultra-sensitively detect antibodies using antigen—DNA conjugates.

Alternatively, the presence or level of a marker of interest can be determined by detecting or quantifying the amount of the purified marker. Purification of the marker can be achieved, for example, by high pressure liquid chromatography (HPLC), alone or in combination with mass spectrometry (e.g., MALDI/MS, MALDI-TOF/MS, tandem MS, etc.). Qualitative or quantitative detection of a marker of interest can also be determined by well-known methods including, without limitation, Bradford assays, Coomassie blue staining, silver staining, assays for radiolabeled protein, and mass spectrometry.

The analysis of a plurality of markers may be carried out separately or simultaneously with one test sample. For separate or sequential assay of markers, suitable apparatuses include clinical laboratory analyzers such as the ElecSys (Roche), the AxSym (Abbott), the Access (Beckman), the AD VIA®, the CENTAUR® (Bayer), and the NICHOLS ADVANTAGE® (Nichols Institute) immunoassay systems. Particularly useful physical formats comprise surfaces having a plurality of discrete, addressable locations for the detection of a plurality of different markers. Such formats include protein microarrays, or “protein chips” and certain capillary devices (see, e.g., U.S. Pat. No. 6,019,944). In these embodiments, each discrete surface location may comprise antibodies to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection.

As for the format of the test, it is understood that other diagnostic test devices may be adapted for the use of the present invention. For example, a strip test assay is well known in the art where the sample is applied to one end of the strip and the fluid migrates by capillary action up to the test zone. A sample can be any solution including body fluids (e.g. whole blood, serum or plasma, urine and the like).

The test zone contains an immobilized bound reagent for the detection of the desired analyte. Reagents can be immobilized via any suitable technique as will be apparent to those skilled in the art. Direct attachment methods include nondiffusive adsorption, nondiffusive absorption, attachment to microparticles that are themselves entrapped in the appropriate position, and covalent binding, such as by use of cyanogen bromide, carbonyl diimidazole, or glutaraldehyde. If the test result is positive, then the test zone will display a positive result; i.e., it will change color, altering the bar code by “adding” an additional stripe. In a similar embodiment, the test zone might be configured such that detection of an analyte will result in disappearance of the test zone stripe, such that the data encoded in the bar code is changed as well.

In general, the sample is suspected of containing an analyte. An analyte will typically be one member of a specific binding pair, while the test zone of the strip test will contain a second member of a specific binding pair. A member of a specific binding pair can include, for example, substances such as antigens, antibodies, receptors, peptides, proteins, ligands, single-stranded and double-stranded DNA, oligonucleotides, cDNA, mRNA, RNA, and the like. The analyte can be monovalent (monoepitopic) or polyvalent (polyepitopic), synthetic or natural, antigenic or haptenic, and may be a single compound or plurality of compounds which share at least one common epitopic or determinant site. The detection of a specific binding pair may occur simultaneously with the test, or may occur in one or more subsequent steps, depending on the test.

The formation of a specific binding pair between the analyte of interest and the reagent immobilized in the test zone may be detected by visual readout or machine-assisted readout. The detectable indication can be a color change, if a visible result is desired. In other embodiments, the detectable indication is created by enzymes, fluorophores, chromophores, radioisotopes, dyes, colloidal gold, colloidal carbon, latex particles, and chemiluminescent agents. In some embodiments, the detectable indication is not visible to the eye, but is detected by suitable equipment. Such is the case when the specific binding pair is fluorescent, or radioactive.

Methods to detect antibodies, including autoantibodies, are known, for example using immunodiffusion methods. Immunodiffusion techniques can be useful in analyzing a large number of biological components, including antibodies, proteins, enzymes and nucleic acids, depending on the particular binding agents employed. For example, where the analyte is an antibody, typical binding agents are antigens, and vice versa. Such techniques involve screening for the presence of an analyte by diffusing a solution suspected of containing the analyte through a support and by diffusing the antigen. The analyte contained in the sample eventually reacts with the antigen in solution producing a complex analyte-antigen. This complex between the antigen and analyte can be detected by a variety of indicators. For example, sandwich immunoassay techniques involve the formation of a three-member complex of antigen-analyte-label that can be detected via visual, radioactive, spectroscopic, or other methods. In yet another example, the complex analyte-antigen can create zones of precipitation resulting from immunodiffusion that can be subjected to direct quantitative measurements such as quantitative photooptical measurements of the light intensity.

Enzyme-linked immunosorbent assay (ELISA) methods are described above. For detection of the endogenous antibodies of the invention, for example, antigens to the endogenous antigens are attached to a surface. Then, the sample is contacted with the antigens, which act as bait to bind the endogenous antibodies, and a further specific antibody is applied over the surface, which can bind to the endogenous antibodies. This antibody is linked to an enzyme, and, in the final step, a substance containing the enzyme's substrate is added. The subsequent reaction produces a detectable signal, most commonly a color change in the substrate.

Western blot techniques can be useful in analyzing a large number of biological components. For example, an antigen or an antigenic mixture of interest is solubilized, usually with sodium dodecyl sulfate (SDS), urea, and, alternatively, with reducing agents such as 2-mercaptoethanol or the likes. Following solubilization, the material is separated on a polyacrylamide gel by electrophoresis and the antigens are then electrophoretically transferred to a support, where they are bound irreversibly. The membrane is exposed to the sample suspected of containing the analyte. The analyte contained in the sample eventually reacts with the antigen producing a complex analyte-antigen. The complex between the antigen and analyte can be detected by a variety of indicators such as a labeled detected antibody. In another example, the antigen is placed in contact with the sample suspected of containing the analyte. This complex is then run on a non-denaturing polyacrylamide gel by electrophoresis and the antigens are then electrophoretically transferred to a support, where it is bound irreversibly. The complex between the antigen and analyte can be detected by a variety of indicators such as a labeled detection antibody. In yet another example, the antigen is placed in contact with the sample suspected of containing the analyte. This complex is then transferred to a support, where it is bound irreversibly. The complex between the antigen and analyte can be detected by a variety of indicators such as a labeled detection antibody.

Anti-idiotypic antibodies techniques can be useful in analyzing a large number of biological components. For example, antibodies that bind IBD-associated antigens are isolated from one or more subjects and injected into a mammal such as mice, goats, rabbit, and the likes. The resulting anti-idiotypic polyclonal or monoclonal antibodies are used in assays to detect antibodies to IBD-associated antigens in subjects. For example, the assay is a competitive method for detecting the present of analyte contained in a sample. The assay includes incubating the antigen with an anti-idiotypic antibody and an unknown amount of analyte present in the sample collected from a subject wherein the antigen is either enzyme labelled or indirectly detected, whereby the presence of analyte in the sample is determined by comparing the extent to which its binding to the antigen is displaced by the addition of the anti-idiotypic antibody with a calibration curve obtained with a known amount of analyte or derivatives thereof.

Techniques based on mobility shift assay can be used to detect and quantify autoantibodies or any other type of antibodies against specific antigens present in any kind of samples. The sample can be subjected to differential separation by using size exclusion chromatography (either regular or high performance liquid chromatography) or any of the methods that relies on different mobility properties. Basically, the sample to be analyzed will be put in contact with the specific antigen which has been labeled with any standard labeling method (i.e. fluorophores, colored substrates, enzymes, or others), and further subjected to size exclusion chromatography or any other method based on the differential physico-properties of free versus bound antigen.

In addition to the above-described assays for determining the presence or level of various markers of interest, analysis of marker mRNA levels using routine techniques such as Northern analysis, reverse-transcriptase polymerase chain reaction (RT-PCR), or any other methods based on hybridization to a nucleic acid sequence that is complementary to a portion of the marker coding sequence (e.g., slot blot hybridization) are also within the scope of the present invention. General nucleic acid hybridization methods are described in Anderson, “Nucleic Acid Hybridization,” BIOS Scientific Publishers, 1999. Amplification or hybridization of a plurality of transcribed nucleic acid sequences (e.g., mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi, P and G. Westley., “DNA Microarrays and Gene Expression: From Experiments to Data Analysis and Modeling,” Cambridge University Press, 2002.

Analysis of the genotype of a marker such as a genetic marker can be performed using techniques known in the art including, without limitation, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry (MALDI-TOF/MS), and sequencing by hybridization. Non-limiting examples of electrophoretic analysis include slab gel electrophoresis such as agarose or polyacrylamide gel electrophoresis, capillary electrophoresis, and denaturing gradient gel electrophoresis. Other methods for genotyping an individual at a polymorphic site in a marker include, e.g., the INVADER® assay from Third Wave Technologies, Inc., restriction fragment length polymorphism (RFLP) analysis, allele-specific oligonucleotide hybridization, a heteroduplex mobility assay, and single strand conformational polymorphism (SSCP) analysis.

Several markers of interest may be combined into one test for efficient processing of a multiple of samples. In addition, one skilled in the art would recognize the value of testing multiple samples (e.g., at successive time points, etc.) from the same patient. Such testing of serial samples can allow the identification of changes in marker levels over time. Increases or decreases in marker levels, as well as the absence of change in marker levels, can also provide useful information to classify IBD or to differentiate between clinical subtypes of IBD.

A panel consisting of one or more of the markers described above may be constructed to provide relevant information related to the approach of the present invention for classifying a patient sample as being associated with IBD or a clinical subtype thereof. Such a panel maybe constructed using 1, 2, 3, 4, 5, 6, 7, 8, 9, 10, 11, 12, 13, 14, 15, 16, 17, 18, 19, 20, 25, 30, 35, 40, or more individual markers. The analysis of a single marker or subsets of markers can also be carried out by one skilled in the art in various clinical settings.

The analysis of markers could be carried out in a variety of physical formats as well. For example, the use of microtiter plates or automation could be used to facilitate the processing of large numbers of test samples. Alternatively, single sample formats could be developed to facilitate treatment and diagnosis in a timely fashion.

In one aspect the invention relates to a kit for the detection of inflammation-associated autoantibodies in a sample comprising:

i. one or more peptide reagents as described above; and
ii. a means for detection of a complex formed between the peptide and an inflammation-associated autoantibody.

The kit may contain ready to use reagents and the test results are advantageously obtained within several hours, e.g., less than six hours. For example, the kit may contain all ready to use reagents including coated plates, negative and positive controls, wash solution, sample diluent, conjugate, TMB and stop solutions. In some embodiments the solid phase of the test is coated with peptide antigen as described above. The peptide antigen can be chemically synthesized or expressed in E. coli or other suitable bacterial expression line. In the method and test kit any known and useful solid phase may be used. For example, MaxiSorp or PolySorp (Thermo Fisher Scientific) may be used and coated by applying a coating buffer which has a pH of, for example, 5, 7 or 9.5. The antigen is applied in a quantity of 0.1, 0.5, 1, 2, 3 or 4 ug/ml. A diluent may be used, for example (i) 0.14 M NaCl, 2.7 mM KCl, Kathon 0.03%, Tween 20 0.1%.; or (ii) 2% MgCl2, 6% Tween20 and 6% AO, 0.5% Casein sodium salt. The detection antibody is diluted, for example 1:10000 or 1:20000.

The method steps will be applied as required and may vary depending to the particular reagents applied. In a one embodiment the conditions and method steps are as follows:

a) Sample (1:10) in sample diluent (MgCl2 2%, AO 6%, Tween20 6%, Casein 0.5%), 100 μl/well;
b) Incubate 1 h, room temperature in humid chamber;
c) 3× wash (phosphate buffered saline with 0.1% Tween20);
d) Conjugate ready-to-use, 100 μl/well;
e) Incubate 1 h, room temperature in humid chamber;
f) 3× wash (phosphate buffered saline with 0.1% Tween20);
g) TMB 100 ul/well, incubate 10 mins, room temperature;
h) Add stop solution (100 μl/well); and

### i) Read out at 450 nm

In some embodiments the sample diluent contains casein sodium salt in a concentration of between 0.1 to 0.55%. For example, the sample diluent may contain 0.5% casein sodium salt and MgCl2, e.g., at a concentration of 2%.

In some embodiments the method of detection and/or the kit, is characterized by the inclusion of specific compounds, the use of particular dilutions of the capturing antigen and/or a particular amount and quality of capturing antigen coated onto the solid support used in the method of detection and the kit of the invention.

In some embodiments, the dilution of the antigen is chosen to be in the coating solution in a concentration of 0.25 to 5 μg/ml, for example, 0.5 to 1 μg/ml. The coating step is, for example, performed at pH 5 to 10, e.g. about 5, 7 or 9.5. The antigen as described in the specification and Examples, in some embodiments is used in amounts of 0.1, 0.5, 1, 2, or 4 μg/ml, e.g., 1 μg/ml.

In some embodiments, the method of detection and kit contains Tween, e.g. a Tween 20, or a comparable substance, e.g., a detergent with comparable characteristics. For example, the substance is contained in an amount of 0.05 to 0.5%, for example 0.1 to 0.2%.

In some embodiments the wash solution of the coating step contains NaCl 0.14M, KCl 2.7 mM; Kathon 0.03%, Tween20 0.1%, sample diluent comprises MgCl₂ 2%, aminoxid (AO) 6%, Tween20 6% and 0.5% casein. For example, the conjugate (where the patient is a human, the anti-human Ab conjugate) is used in a dilution of 1:10 000 to 1:30 000, e.g., 1:20 000 in a conjugate stabilizing buffer as a ready to use format.

The immunoassays described herein may be configured in a reagent impregnated test strip in which a specific binding assay is performed in a rapid and convenient manner with a minimum degree of skills and involvement.

For example, the test strip is prepared with one or multiple detection zones in which the specific binding reagents (labeled or unlabeled) for an analyte suspected of being in the sample is immobilized. A sample of serum (or any other body fluid) is applied to one portion of the test strip comprising of a dry carrier (such as nitrocellulose or any other bibulous, porous or fibrous material capable of absorbing liquid rapidly) and is allowed to permeate through the strip material with the aid of an eluent such as phosphate buffer or the like. The sample progresses through the detection zone wherein a specific binding reagent has been immobilized.

In certain embodiments, the immobilized agents can comprise an antigen or a plurality of antigens that will bind to certain inflammation-associated autoantibodies present in a sample from a patient having an inflammatory condition, for example a calprotectin or antigenic fragment thereof, comprising at least 10 (e.g., at least 20, e.g., at least 30) consecutive amino acids in a sequence from a wild type calprotectin, e.g., human calprotectin, e.g., from SEQ ID NO 19 or 20, and/or an integrin or antigenic fragment thereof, comprising at least 10 (e.g., at least 20, e.g., at least 30) consecutive amino acids in a sequence from a wild type integrin, e.g. from human integrin, e.g., from SEQ ID NO 21, 22 or 23.

In some embodiments, the immobilized agents will further comprise a positive control, for example, a common antigen that will bind antibodies present in the serum or all or nearly all the patient species.

The inflammation-associated autoantibodies and/or IBD-associated antibodies present in the sample can therefore become bound within the detection zone to the immobilized antigen. The antibody thus bound is capable of participating in a sandwich reaction where a second labeled binding reagent (e.g., a secondary antibody covalently linked to horseradish peroxidase or alkaline phosphatase or the like) is applied that operates as a specific binding partner for the given analyte. The labeled reagent, the analyte (if present) and the immobilized unlabeled specific binding reagent cooperate together in a sandwich reaction. The two binding reagents must have specificities for different epitopes on the analyte. The color generated at the detection zone can be read by eye or using a light refractometer. A quantitative variant of the test can be developed by testing mixtures of specific binding reagent. Alternatively, polymer particles (e.g., latex) can be colored and sensitized with reagents (e.g., proteinaceous antigens or antibodies) and used to detect specific analytes present in samples that have been deposited in detecting zones. Color development at test site may be compared with color of one or more standards or internal controls.

Broadly, the strip test cell and process of this example can be used to detect any analyte which has heretofore been assayed using known immunoassay procedures, or known to be detectable by such procedures, using polyclonal or monoclonal antibodies or other proteins comprising binding sites for such analytes. Various specific assay protocols, reagents, and analytes useful in the practice of the example invention are known per se, see, e.g., U.S. Pat. No. 4,446,232 and U.S. Pat. No. 4,868,108.

### IV. Statistical Algorithms

In some aspects, the present invention provides methods, systems, and code for classifying whether a patient sample is associated with an inflammatory condition such as IBD using a statistical algorithm or process to classify the sample as an inflammatory sample or non-inflammatory sample, in other aspects, the present invention provides methods, systems, and code for classifying whether a sample is associated with a clinical subtype of an inflammatory condition, (e.g. differentiating between IBD which is Crohn's disease vs. ulcerative colitis for example) using a statistical algorithm or process to classify the sample. The statistical algorithms or processes independently can comprise one or more learning statistical classifier systems. As described herein, a combination of learning statistical classifier systems advantageously provides improved sensitivity, specificity, negative predictive value, positive predictive value, and/or overall accuracy for classifying whether a sample is associated with IBD or a clinical subtype thereof.

### V. Detecting Markers for Inflammatory Conditions In Mammals

In one embodiment, the invention provides a method (Method A) for detecting the presence and/or level of one or more inflammation-associated autoantibodies, e.g., endogenous antibodies to an inflammatory marker, e.g., selected from autoantibodies to calprotectin, autoantibodies to integrins, autoantibodies, autoantibodies to lactoferritin, and autoantibodies to C-reactive protein, in a sample obtained from a human patient, wherein the sample is selected from antibody-containing physiologic materials, e.g., selected from one or more of whole blood, saliva, mucus secretions, serum, plasma, stool, and intestinal tissue; said method comprising the steps of

a. Contacting an antigen bound to a substrate or detectable label with said sample, and detecting the binding of said one or more inflammation-associated autoantibodies to said antigen; and/or

b. Contacting a labeled antibody with said sample, wherein the labeled antibody specifically binds human immunoglobulin, and detecting binding of the labeled antibody to said one or more inflammation-associated autoantibodies; and

c. Optionally, classifying said sample as an inflammation sample or non-inflammation sample, wherein the presence or level of the one or more inflammation-associated autoantibodies, separately or in combination, correlates with the presence of an inflammatory condition.

A.1. Any preceding method wherein the patient exhibits clinical symptoms of an inflammatory condition, e.g., clinical symptoms of IBD, e.g., one or more of the following symptoms:

a. Blood in the stool;

b. Elevated levels of fecal calprotectin;

c. Elevated levels of fecal lactoferrin;

d. Anemia;

e. Diarrhea;

f. Vomiting;

g. Inappetence;

h. Fever;

i. Persistent pain; or

j. Significant recent weight loss.

A.2. Any preceding method wherein the inflammation-associated autoantibody is selected from autoantibodies to calprotectin, autoantibodies to integrins, autoantibodies to lactoferritin, autoantibodies to C-reactive protein, and combinations thereof, for example, an autoantibody to calprotectin and/or to an integrin, for example, wherein the inflammation-associated autoantibody is an autoantibody to calprotectin or wherein the inflammation-associated autoantibody is an autoantibody to an integrin.
A.3. Any preceding method wherein the inflammation associated autoantibody is an IgA.
A.4. Any preceding method wherein the inflammation associated autoantibody is a secretory IgA.
A.5. Any preceding method wherein the inflammation associated autoantibody is a serum IgA.
A.6. Any preceding method wherein the sample comprises saliva.
A.7. Any preceding method wherein the sample comprises whole blood.
A.8. Any preceding method wherein the presence of the inflammation associated autoantibody indicates a chronic inflammatory condition.
A.9. Any preceding method wherein the presence of the inflammation associated autoantibody indicates IBD.
A.10. Any preceding method wherein the presence, severity and/or type of an inflammatory condition in the patient is associated with antibody class switching from IgG to IgA, for example, wherein the proportion of IgG autoantibodies relative to IgA autoantibodies to the same antigen is higher in healthy animals and lower in animals with an inflammatory condition.
A.11. Any preceding method further comprising applying a statistical algorithm to said presence or level of one or more inflammation-associated autoantibodies to obtain a diagnostic or prognostic profile for said patient, wherein the presence or relative levels of particular inflammation-associated autoantibodies correlates with the presence, type or severity of inflammation.
A.12. Any preceding method wherein the antigen bound to a substrate or a detectable label is

a. an isolated peptide, which is a calprotectin or antigenic fragment thereof, comprising at least 10 (e.g., at least 20, e.g., at least 30) consecutive amino acids in a sequence from a wild type calprotectin, e.g. from a human calprotectin, e.g., from SEQ ID NO 19 or 20, wherein the calprotectin or antigenic fragment thereof is bound to one or more of a label, a purification tag, a solid substrate, or another protein or fragment thereof, for example another calprotectin or fragment thereof or an integrin or fragment thereof; for example, wherein the calprotectin or antigenic fragment thereof is bound to a poly-histidine tag, for example, a N-terminal hexa-histadine tag; and/or

b. An isolated peptide which is an integrin or antigenic fragment thereof, comprising at least 10 (e.g., at least 20, e.g., at least 30) consecutive amino acids in a sequence from a wild type integrin, e.g. from a human integrin, e.g., from SEQ ID NO 21, 22 or 23, wherein the integrin or antigenic fragment thereof is bound to one or more of a label, a purification tag, a solid substrate, or another protein or fragment thereof, for example, a calprotectin or fragment thereof or another integrin or fragment thereof; for example, wherein the integrin or antigenic fragment thereof is bound to a poly-histidine tag, for example a N-terminal hexa-histadine tag.

A.13. Any preceding method wherein the antigen bound to a substrate or a detectable label is a calprotectin S100A8/S100A9 heterodimer.
A.14. Any preceding method wherein the antigen bound to a substrate or a detectable label is an integrin alpha-4/beta-7 heterodimer.
A.15. Any preceding method wherein the antigen bound to a substrate or a detectable label is a fusion peptide comprising one or more antigenic fragments of a calprotectin S100A8 monomer, e.g., a fragment comprising at least 10 (e.g., at least 20, e.g., at least 30) consecutive amino acids in a sequence from SEQ ID NO: 19, and one or more antigenic fragments of a calprotectin S100A9 monomer, e.g., a fragment comprising at least 10 (e.g., at least 20, e.g., at least 30) consecutive amino acids in a sequence from SEQ ID NO: 20, wherein the fragments are linked by one or more amino acid spacer sequences.
A.16. Any preceding method wherein the antigen bound to a substrate or a detectable label is a fusion peptide comprising one or more antigenic fragments of an integrin α (alpha) subunit, e.g., comprising at least 10 (e.g. at least 20, e.g., at least 30) consecutive residues from an integrin α (alpha) subunit, e.g., from SEQ ID NO: 21, and one or more antigenic fragments of an integrin β (beta) subunit, e.g., comprising at least 10 (e.g. at least 20, e.g., at least 30) consecutive residues from an integrin β (beta) subunit, e.g., from SEQ ID NO: 22 or 23, wherein the fragments are linked by one or more amino acid spacer sequences.
A.17. Any preceding method further comprising detecting the presence or level in the sample of one or more additional IBD-associated endogenous antibodies, e.g., selected from the group consisting of an anti-PMN antibody, anti-yeast antibody, antimicrobial antibody, and combinations thereof, in the sample.
A.18. Any preceding method further comprising detecting the presence or level in the sample of one or antibodies associated with food sensitivity, e.g., antibodies to antigens from wheat, corn, soy, dairy, milk, eggs, gliadin, zein, amylase inhibitor, or tissue transglutaminase (e.g. TTG2, or TG3).
A.19. Any preceding method further comprising detecting the presence and/or level of one or more endogenous antibodies in the sample obtained from the patient, wherein the endogenous antibodies are selected from one or more of endogenous antibodies to one or more proteins associated with food sensitivity, e.g., selected from one or more of gliadin, zein, amylase inhibitor, or tissue transglutaminase (e.g. TTG2, or TG3), and/or detecting the presence and/or level of one or more endogenous antibodies associated with gastrointestinal infection, including endogenous antibodies to polymorphonuclear leukocytes (PMNs or granulocytes, including neutrophil granulocytes) and/or endogenous antibodies to microbes found in the gut, for example bacterial OmpC, flagellin; e.g., wherein detecting the presence of level of said endogenous antibodies comprises

a. contacting one or more antigens with said sample, wherein the one or more antigens are specific for the endogenous antibody of interest, and wherein the one or more antigens are bound to a substrate or detectable label, and

b. detecting the binding of said one or more one or more endogenous antibodies associated with inflammation to the one or more antigens,

c. and optionally, classifying said sample as classifying the sample, e.g.,
i. as positive or negative for food sensitivity, wherein the presence or level of the one or one or more endogenous antibodies associated with food sensitivity, separately or in combination, correlates with the presence of food sensitivity, and/or
ii. as positive or negative for infection, wherein the presence or level of one or more endogenous antibodies associated with gastrointestinal infection, separately or in combination, correlates with the presence of gastrointestinal infection.
   A.20. Any preceding method further comprising applying a statistical algorithm to said the presence or level of one or more inflammation-associated autoantibodies in combination with the presence or level of one or more one or more additional IBD-associated endogenous antibodies, e.g., selected from the group consisting of an anti-PMN antibody, anti-yeast antibody, antimicrobial antibody, and combinations thereof in the sample.
   A.21. Any preceding method wherein said patient is diagnosed with Crohn's disease or ulcerative colitis.
   A.22. Any preceding method wherein the sample is additionally assayed for the presence or level of one or more additional IBD-associated endogenous antibodies are selected from the group consisting of an anti-PMN antibody, anti-yeast antibody, antimicrobial antibody, and combinations thereof in said.
   A.23. The foregoing method wherein the one or more additional IBD-associated endogenous antibodies comprise

a. anti-PMN antibody selected from the group consisting of an anti-PMN antibody (APMNA), perinuclear anti-PMN antibody (pAPMNA), and combinations thereof; and/or

b. anti-yeast antibody selected from the group consisting of anti-yeast immunoglobulin A (AYA-IgA), anti-yeast immunoglobulin G (AYA-IgG), anti-yeast immunoglobulin M (AYA-IgM) and combinations thereof; and/or

c. antimicrobial antibody selected from the group consisting of an anti-outer membrane protein C (ACA) antibody, anti-flagellin antibody (AFA), and combinations thereof.

A.24. Any of Method A.17, et seq., wherein said the one or more additional IBD-associated endogenous antibodies are selected from APMNA, pAPMNA, AYA-IgA, AYA-IgG, ACA, or AFA.
A.25. Any of Method A.17, et seq., wherein said the one or more additional IBD-associated endogenous antibodies are IgA antibodies.
A.26. Any preceding method wherein the immunoassay to detect the presence or level of the one or more inflammation associated autoantibodies is an enzyme-linked immunosorbent assay (ELISA).
A.27. Any preceding method wherein the immunoassay to detect the presence or level of the one or more inflammation-associated autoantibodies is an agglutination-PCR (ADAP).
A.28. Any preceding method, wherein the immunoassay to detect the presence or level of the one or more inflammation-associated autoantibodies is an immunohistochemical assay.
A.29. Any preceding method, wherein the immunoassay to detect the presence or level of the one or more inflammation-associated autoantibodies is an immunoflourescence assay.
A.30. Any preceding method, wherein said sample is selected from the group consisting of saliva, serum, plasma, and whole blood.
A.31. Any preceding method, wherein the step of classifying said sample as an inflammation sample or non-inflammation sample is carried out using a statistical algorithm selected from the group consisting of a classification and regression tree, boosted tree, neural network, random forest, support vector machine, general chi-squared automatic interaction detector model, interactive tree, multiadaptive regression spline, machine learning classifier, and combinations thereof.
A.32. Any preceding method, comprising: (a) determining the presence or level of at least one inflammation-associated autoantibody, (b) optionally determining the presence or level of at least one marker selected from the group consisting of an anti-polymorphonuclear leukocyte (PMN) antibody, antimicrobial antibody, calprotectin and combinations thereof in the sample; and (c) classifying the sample as an inflammation sample or non-inflammation sample using a statistical algorithm based upon the presence or level of at least one marker.
A.33. Any preceding method wherein the one or more antigens bound to a substrate or detectable label is any of Reagent A, as hereinafter described.
A.34. Any preceding method further comprising detecting the presence or level of detecting the presence and/or level of one or more endogenous antibodies associated with inflammatory bowel disease (IBD-associated antibodies), e.g., wherein the one or more IBD-associated antibodies are selected from the group consisting of an anti-PMN antibody, anti-yeast antibody, antimicrobial antibody, and combinations thereof.
A.35. Any preceding method wherein the one or more antigens are bound to one or more substrates, wherein the substrates comprise one or more microwell plates, such that where detecting binding to different antigens is desired, the different antigens are on different microwell plates or in different wells of the same microwell plate; e.g. wherein the microwell plate is a flat plate or strip with multiple sample wells, e.g., 6, 24, 96, 384 or 1536 sample wells, e.g., wherein each well of the microwell plate has a volume between 10 nl to 1 ml, for example between 50 μl and 500 μl.
A.36. Any preceding method, wherein the one or more antigens are bound to one or more substrates, comprising the steps of

a. Affixing the one or more antigens to their respective substrates,

b. Blocking any uncoated surfaces of the substrates with protein, e.g., bovine serum albumin

c. Exposing the antigens to the sample to allow formation of antigen-antibody complexes,

d. Exposing the antigen-antibody complexes thus formed to the labeled antibody to a labeled antibody that binds the immunoglobulin, e.g., IgA, from the patient, e.g., horseradish peroxidase (HRP)-anti-IgA antibody

e. Detecting binding of the labeled antibody to the antigen-antibody complexes, e.g., wherein the substrate is washed with buffer after each of steps a-d.

A.37. Any preceding method comprising classifying the sample from the patient as “consistent” with an inflammatory condition in the patient, e.g., inflammatory bowel disease (IBD), or “not consistent” with the inflammatory condition, wherein the presence and/or level of IgA in the sample that binds to the one or more antigens, separately or in combination, correlates with the presence of the inflammatory condition in the patient.
A.38. Any preceding method wherein the antigens used comprise one or more of Reagents 1, et seq.

In another embodiment, the invention provides a method of diagnosing an inflammatory condition comprising detecting the presence and/or level of the one or more IBD-associated antibodies, separately or in combination, in accordance with any of Method A, et seq.

In another embodiment, the invention provides a method of classifying whether a patient is associated with a clinical subtype of inflammation, the method comprising: (a) determining the presence or level of at least one inflammation-associated autoantibody, (b) optionally determining the presence or level of at least one marker selected from the group consisting of an anti-polymorphonuclear leukocyte (PMN) antibody, antimicrobial antibody, calprotectin and combinations thereof in the sample; and (c) classifying the sample lymphoplasmacytic (LPE) IBD, eosinophilic gastroenterocolitis (EGE) IBD or granulomatous (GE) IBD or non-IBD sample using a statistical algorithm based upon the presence or level of the at least one marker; e.g. using any of Method A, et seq.

In another aspect, the present invention provides a method for monitoring the progression or regression of inflammation in a patient the method comprising: (a) determining the presence or level of at least one inflammation-associated autoantibody, (b) optionally determining the presence or level of at least one marker selected from the group consisting of an anti-polymorphonuclear leukocyte (PMN) antibody, antimicrobial antibody, calprotectin and combinations thereof in the sample; and (c) determining the presence or severity of inflammation using a statistical algorithm based upon the presence or level of the at least one marker; e.g., using any of Method A, et seq.

In a related aspect, the present invention provides a method for monitoring drug efficacy in a patient receiving drugs useful for treating inflammation, the method comprising: (a) determining the presence or level of at least one inflammation-associated autoantibody, (b) optionally determining the presence or level of at least one marker selected from the group consisting of an anti-polymorphonuclear leukocyte (PMN) antibody, antimicrobial antibody, calprotectin and combinations thereof in the sample; and (c) determining the presence or severity of inflammation using a statistical algorithm based upon the presence or level of the at least one marker; e.g. using any of Method A, et seq.

In some embodiments, the invention provides a method of detecting multiple types of endogenous antibody in a patient, including detecting endogenous antibody to inflammatory markers, e.g., according to any of Method A, et seq. and also detecting other endogenous antibodies, e.g., to food antigens, and/or bacterial antigens. Examples of such other endogenous antibodies, which in some embodiments can be detected in the same patient as the endogenous antibodies to inflammatory markers, include anti-neutrophil cytoplasmic antibody (ANCA), anti-Saccharomyces cerevisiae immunoglobulin A (ASCA-IgA), anti-Saccharomyces cerevisiae immunoglobulin G (ASCA-IgG), an anti-outer membrane protein C (anti-OmpC) antibody, an anti-flagellin antibody, an anti-I2 antibody, and a perinuclear anti-neutrophil cytoplasmic antibody (pANCA), e.g. as described in US 20060154276A1; WO 2014053996, and US 20100094560A1, all incorporated herein by reference.

Detecting combinations of IgA to different antigens is especially valuable for differential diagnosis among gastrointestional disorders, for example IBD, infection and food sensitivity. For example, in a particular embodiment, the method comprises detecting the absolute and relative levels of endogenous IgA to calprotectin (e.g., as described in Method A, et seq.) and also detecting endogenous antibodies to gliadin (indicating a food sensitivity to wheat), and OmpC from an intestinal bacterial strain (indicating infection and/or permeability of the intestinal wall), and the detection of the presence and relative levels of such combinations of endogenous IgA markers provides particularly useful information for diagnosis of gastrointestinal disorders. For example, the invention provides a method (Method A′) for detecting the presence and/or level of combinations of at least the following endogenous IgA markers in serum obtained from a patient:

a. endogenous IgA to gliadin;

b. endogenous IgA to a bacterial outer membrane protein C (OmpC),

c. endogenous IgA to calprotectin, and said method comprising the steps (carried out simultaneously or sequentially in any order) of

a1) contacting said serum with a gliadin antigen bound to a substrate, wherein the gliadin antigen comprises one or more antigenic sequences from gliadin;
a2) detecting the binding of endogenous IgA markers to the gliadin antigen using a labeled antibody which binds human IgA;
b1) contacting said serum with an OmpC antigen bound to a substrate, wherein the OmpC antigen comprises one or more antigenic sequences from an OmpC of an intestinal bacteria strain;
b2) detecting the binding of endogenous IgA markers to the OmpC antigen using a labeled antibody which binds human IgA;
c1) contacting said serum with a calprotectin antigen bound to a substrate, wherein the calprotectin antigen comprises one or more antigenic sequences from calprotectin;
c2) detecting the binding of endogenous IgA markers to the calprotectin antigen using a labeled antibody which binds human IgA; e.g., in accordance with any of Methods A, et seq.; for example:

A′-1. Method A′ wherein the gliadin antigen is an isolated peptide comprising one or more sequences from gliadin that do not contain protease cleavage sites for proteases in gastric fluid but not comprising sequences from gliadin that do contain such sites.

A′-2. Any foregoing method wherein the calprotectin antigen comprises a calprotectin S100A8 monomer region (e.g., a region comprising at least 10, e.g. at least 20, e.g. at least 30, consecutive amino acid residues from SEQ ID NO 19), and a calprotectin S100A9 monomer region (e.g., a region comprising at least 10, e.g. at least 20, e.g. at least 30, consecutive amino acid residues from SEQ ID NO 20), wherein the regions are linked by a linker sequence.

A′-3. Any foregoing method wherein the calprotectin antigen is a calprotectin S100A8/S100A9 heterodimer.

A′-4. Any foregoing method wherein the gliadin antigen, the OmpC antigen, and the calprotectin antigen each comprises a polyhistidine tag.

A′-5. Any foregoing method wherein the polyhistadine tag comprises SEQ ID NO: 18.

A′-6. Any foregoing method wherein the substrates for the gliadin antigen, the OmpC antigen, and calprotectin antigen comprise one or more microwell plates, and wherein the gliadin antigen, the OmpC antigen, and calprotectin antigen are on different microwell plates or in different wells of the same microwell plate.

A′-7. Any foregoing method comprising the steps of
a. affixing the gliadin antigen, the OmpC antigen, and the calprotectin antigen to their respective substrates,
b. blocking any uncoated surfaces of the substrates with protein,
c. exposing the antigens to the serum sample to allow formation of antigen-antibody complexes between the antigen and endogenous IgA,
d. exposing the antigen-IgA complexes thus formed to the labeled antibody,
e. detecting binding of the labeled antibody to the antigen-IgA complexes.

A′-8. The foregoing method wherein the substrate is washed with buffer after each of steps a-d.

A′-9. Any foregoing method of claim 1 wherein the labeled antibody is an anti-human IgA antibody linked to an enzyme.

A′-10. Any foregoing method wherein the labeled antibody is an anti-human IgA antibody linked to an enzyme and the steps a2, b2, and c2 are carried out by (i) contacting the endogenous IgA bound to antigen with the labeled antibody, (ii) providing a substrate for the enzyme, and (iii) measuring the increase in optical density caused by the reaction of the enzyme with the substrate for the enzyme, wherein the increase in optical density correlates with the presence and amount of endogenous IgA bound to antigen.

A′-11. Any foregoing method wherein the enzyme is horseradish peroxidase (HRP) and the substrate is 3,3′,5,5′-Tetramethylbenzidine (TMB).

The invention further provides a method of diagnosing and differentiating among inflammation, gastrointestinal infection, and food sensitivity in a patient exhibiting symptoms of gastrointestinal disorder, comprising

a. detecting endogenous IgA to gliadin; endogenous IgA to a bacterial outer membrane protein C (OmpC), and endogenous IgA to calprotectin in the serum of the patient, e.g., in accordance with any of Methods A, et seq. or Methods A′, et seq., and

b. diagnosing the presence of inflammation when relatively high levels of endogenous IgA to calprotectin are detected in the serum of the patient,

c. diagnosing gastrointestinal infection when relatively high levels of endogenous IgA to a bacterial outer membrane protein C (OmpC) are detected in the serum of the patient, and

d. diagnosing food sensitivity when relatively high levels of endogenous IgA to gliadin are detected in the serum of the patient.

A method of treating gastrointestinal disorders in a patient in need thereof, comprising

a. diagnosing the patient in accordance with the foregoing method, and

b. when inflammation is diagnosed, administering an effective amount of a drug selected from anti-inflammatory drugs, immunosuppressive drugs, and combinations thereof to said patient,

c. when gastrointestinal infection is diagnosed, administering an effective amount of antibiotics to said patient, and

d. when food sensitivity is diagnosed, placing the patient on a restricted diet, e.g., a gluten-free diet or hypoallergenic diet.

The invention further provides a kit for use in accordance with any of Methods A′, comprising

a. a gliadin antigen, wherein the gliadin antigen comprises one or more antigenic sequences from gliadin;

b. an OmpC antigen, wherein the OmpC antigen comprises one or more antigenic sequences from bacterial OmpC;

c. a calprotectin antigen, wherein the calprotectin antigen comprises one or more antigenic sequences from calprotectin; and

d. a labeled antibody which binds IgA.

In another embodiment, the invention provides a reagent (Reagent A) comprising an amino acid sequence from one or more of

a. An isolated peptide which is a calprotectin or antigenic fragment thereof, comprising at least 10 (e.g., at least 20, e.g., at least 30) consecutive amino acids in a sequence from a wild type calprotectin, e.g., from a human calprotectin, wherein the calprotectin or antigenic fragment thereof is bound to one or more of a label, a purification tag, a solid substrate, or another protein or fragment thereof, for example another calprotectin or fragment thereof or an integrin or fragment thereof; for example, wherein the calprotectin or antigenic fragment thereof is bound to a poly-histidine tag, for example a N-terminal hexa-histadine tag, optionally further comprising one or more residues to enhance solubility, e.g., an N-terminal sequence in accordance with SEQ ID NO: 15 or 18; and

b. An isolated peptide which is an integrin or antigenic fragment thereof, comprising at least 10 (e.g., at least 20, e.g., at least 30) consecutive amino acids in a sequence from a wild type integrin, e.g. from a human integrin, wherein the integrin or antigenic fragment thereof is bound to one or more of a label, a purification tag, a solid substrate, or another protein or fragment thereof, for example, a calprotectin or fragment thereof or another integrin or fragment thereof; for example, wherein the integrin or antigenic fragment thereof is bound to a poly-histidine tag, for example a N-terminal hexa-histadine tag, optionally further comprising one or more residues to enhance solubility, e.g., an N-terminal sequence in accordance with SEQ NO: 15 or 18.

For example, in some embodiments, Reagent A is

a. a fusion protein comprising a calprotectin S100A8 monomer region, e.g., with sequence comprising at least 20 amino acid residues in sequence from a human calprotectin S100A8 monomer (e.g. from SEQ ID NO. 19) and a calprotectin S100A9 monomer region, e.g., with sequence comprising at least 20 amino acid residues in sequence from a human calprotectin S100A9 monomer (e.g., from SEQ ID NO 20), wherein the regions are linked by a linker sequence, optionally further comprising a polyhistidine sequence and one or more additional residues to enhance solubility, e.g., comprising an N-terminal sequence according to SEQ ID NO 15 or 18; or

b. a fusion peptide comprising an integrin α (alpha) subunit region, comprising at least 20 amino acid residues in sequence from a human integrin α (alpha) subunit (e.g., from an alpha-4 subunit, e.g., from SEQ ID NO: 21), and an integrin β (beta) subunit region, comprising at least 20 amino acid residues in sequence from a human integrin ₃ (beta) subunit (e.g., from a beta-1 subunit, e.g., from SEQ ID NO: 22; or from a beta-7 subunit, e.g., from SEQ ID NO: 23), wherein the regions are linked by a linker sequence, optionally further comprising a polyhistidine sequence and one or more additional residues to enhance solubility, e.g., comprising an N-terminal sequence according to SEQ ID NO 15 or 18.

Linker sequences may, for example, comprise sequences of 10-30, e.g., about 15, amino acid residues, e.g. non-charged amino acid residues, for example glycine and serine residues, e.g., a (Gly₄Ser)ₙ linker, where n is an integer 2 through 5, e.g. 3.

In another embodiment the invention provides a diagnostic kit comprising a reagent according to Reagent A; for example, a diagnostic kit for the detection of inflammation-associated antibodies in a sample from patient, the kit comprising: (i) one or more reagents of Reagent A as described above; and (ii) means for detection of a complex formed between the reagent and an inflammation-associated autoantibody. In some embodiments, the diagnostic kit is an ELISA assay. In some embodiments the kit is a strip assay, wherein antigens, e.g., according to Reagent 1, are bound to specific regions of the strip. In some embodiments, the diagnostic kit is an Agglutination-PCR (ADAP) kit.

In another embodiment the invention provides the use of any reagent as described in Reagent A in the manufacture of a kit or component of a kit for carrying out a diagnostic method according to any of Methods A, et seq.

In another embodiment, the invention provides any reagent described in Reagent A for use in diagnosis, e.g., diagnosis of inflammation in a patient, e.g., in a diagnostic method according to any of Methods A, et seq.

In another embodiment, the invention provides a complex comprising an antigen, an endogenous inflammation-associated antibody bound to the antigen, and a labeled antibody bound to the inflammation-associated antibody, for example wherein the antigen is a reagent according to Reagent A, as hereinbefore described.

In another embodiment, the invention provides a bacterial expression construct comprising a promoter operably linked to an open reading frame encoding one or more of comprising at least 10 (e.g., at least 20, e.g., at least 30) consecutive amino acids in a sequence from a wild type calprotectin, e.g. from a human calprotectin, and/or comprising at least 10 (e.g., at least 20, e.g., at least 30)consecutive amino acids in a sequence from a wild type integrin, e.g. from a human integrin, each optionally linked to an additional sequence, e.g. a polyhistidine tag; wherein the promoter and the open reading frame are heterologous to one another, i.e., wherein the promoter and the open reading frame are not operably linked in nature.

In another embodiment, the invention provides a bacterial cell line, for example an E. coli line, comprising the bacterial expression construct of the preceding paragraph.

### VI. Therapy and Therapeutic Monitoring

Once a patient sample has been classified as an inflammation sample, for example, once a patient sample has been classified as IBD, the methods, systems, and code of the present invention can further comprise administering to the individual a therapeutically effective amount of a drug useful for treating one or more symptoms associated with the particular inflammatory condition, for example, IBD or the IBD subtype. For therapeutic applications, the drug can be administered alone or co-administered in combination with one or more additional anti-inflammatory or anti-IBD drugs and/or one or more drugs that reduce the side-effects associated with the anti-inflammatory or anti-IBD drug.

Anti-inflammatory or anti-IBD drugs can be administered with a suitable pharmaceutical excipient as necessary and can be carried out via any of the accepted modes of administration. Thus, administration can be, for example, intravenous, topical, subcutaneous, transcutaneous, transdermal, intramuscular, oral, buccal, sublingual, gingival, palatal, parenteral, intradermal, intranasal, rectal, vaginal, or by inhalation. By “co-administer” it is meant that an anti-inflammatory or anti-IBD drug is administered at the same time, just prior to, or just after the administration of a second drug (e.g., another IBD drug, a drug useful for reducing the side-effects of the IBD drug, etc.).

A therapeutically effective amount of an anti-inflammatory or anti-IBD drug may be administered repeatedly, e.g., at least 2, 3, 4, 5, 6, 7, 8, or more times, or the dose may be administered by continuous infusion. The dose may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, that can be delivered in unit dosage forms suitable for simple administration of precise dosages.

As used herein, the term “unit dosage form” includes physically discrete units suitable as unitary dosages for human subjects, each unit containing a predetermined quantity of a drug calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the anti-inflammatory or anti-IBD drug.

Methods for preparing such dosage forms are known to those skilled in the art (see, e.g., Remington's Pharmaceutical Sciences, 18^{th} Ed., Mack Publishing Co., Easton, Pa. (1990). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., Remington's Pharmaceutical Sciences, supra).

Examples of suitable excipients include, but are not limited to, lactose, dextrose, sucrose, sorbitol, mannitol, starches, gum acacia, calcium phosphate, alginates, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates; pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the therapeutically effective dose takes the form of a pill, tablet, or capsule, and thus, the dosage form can contain, along with an IBD drug, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. An IBD drug can also be formulated into a suppository disposed, for example, in a polyethylene glycol (PEG) carrier.

Liquid dosage forms can be prepared by dissolving or dispersing an IBD drug and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. An IBD drug can also be formulated into a retention enema.

For topical administration, the therapeutically effective dose can be in the form of emulsions, lotions, gels, foams, creams, jellies, solutions, suspensions, ointments, and transdermal patches. For administration by inhalation, an IBD drug can be delivered as a dry powder or in liquid form via a nebulizer. For parenteral administration, the therapeutically effective dose can be in the form of sterile injectable solutions and sterile packaged powders. Injectable solutions can be formulated at a pH of from about 4.5 to about 7.5. The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to a patient.

In therapeutic use for the treatment of IBD or a clinical subtype thereof, an IBD drug can be administered at the initial dosage of from about 0.001 mg/kg to about 1000 mg/kg daily. A daily dose range of from about 0.01 mg/kg to about 500 mg/kg, from about 0.1 mg/kg to about 200 mg/kg, from about 1 mg/kg to about 100 mg/kg, or from about 10 mg/kg to about 50 mg/kg, can be used. The dosages, however, may be varied depending upon the requirements of the individual, the severity of IBD symptoms, and the IBD drug being employed. For example, dosages can be empirically determined considering the severity of IBD symptoms in an individual classified as having IBD according to the methods described herein. The dose administered to a patient, in the context of the present invention, should be sufficient to affect a beneficial therapeutic response over time. The size of the dose can also be determined by the existence, nature, and extent of any adverse side-effects that accompany the administration of a particular IBD drug in such patient. Determination of the proper dosage for a particular situation is within the skill of the practitioner. Generally, treatment is initiated with smaller dosages which are less than the optimum dose of the IBD drug. Thereafter, the dosage is increased by small increments until the optimum effect under circumstances is reached. For convenience, the total daily dosage may be divided and administered in portions during the day, if desired.

As used herein, the term “IBD drug” includes all pharmaceutically acceptable forms of a drug that is useful for treating one or more symptoms associated with IBD. For example, the IBD drug can be in a racemic or isomeric mixture, a solid complex bound to an ion exchange resin, or the like. In addition, the IBD drug can be in a solvated form. The term is also intended to include all pharmaceutically acceptable salts, derivatives, and analogs of the IBD drug being described, as well as combinations thereof. For example, the pharmaceutically acceptable salts of an IBD drug include, without limitation, the tartrate, succinate, tartarate, bitartarate, dihydrochloride, salicylate, hemisuccinate, citrate, maleate, hydrochloride, carbamate, sulfate, nitrate, and benzoate salt forms thereof, as well as combinations thereof and the like. Any form of an IBD drug is suitable for use in the methods of the present invention, e.g., a pharmaceutically acceptable salt of an IBD drug, a free base of an IBD drug, or a mixture thereof.

As used herein, an anti-inflammatory drug includes IBD drugs, and drugs for treating other inflammatory conditions, including corticosteroids, NSAIDS, and monoclonal antibodies or soluble receptors binding inflammatory cytokines, for example monoclonal antibodies to TNFα.

For example, suitable drugs that are useful for treating one or more symptoms associated with inflammatory conditions such as IBD or a clinical subtype thereof include, but are not limited to, aminosalicylates (e.g., mesalazine, sulfasalazine, and the like), corticosteroids (e.g., prednisone), thiopurines (e.g., azathioprine, 6-mercaptopurine, and the like), methotrexate, monoclonal antibodies (e.g., infliximab), free bases thereof, pharmaceutically acceptable salts thereof, derivatives thereof, analogs thereof, and combinations thereof. One skilled in the art will know of additional anti-inflammatory or IBD drugs suitable for use in the present invention.

A patient can also be monitored at periodic time intervals to assess the efficacy of a certain therapeutic regimen once a sample from such patient has been classified as an IBD sample. For example, the levels of certain markers change based on the therapeutic effect of a treatment such as a drug. The patient is monitored to assess response and understand the effects of certain drugs or treatments in an individualized approach. Additionally, patients may not respond to a drug, but the markers may change, suggesting that these patients belong to a special population (not responsive) that can be identified by their marker levels. These patients can be discontinued on their current therapy and alternative treatments prescribed.

For example, in another embodiment, the invention provides a method (Method 1) for treating an inflammatory condition in a human patient, comprising detecting the presence and/or level of one or more inflammation-associated autoantibodies the patient in accordance with a method according to any one of Method A, et seq. or Method A′, et seq., and administering to said patient a therapeutically effective amount of a drug useful for treating one or more symptoms associated with the inflammatory condition, for example,

1.1.Method 1 wherein the inflammatory condition is IBD.
1.2.Any of Method 1, et seq. wherein the patient exhibits one or more clinical symptoms of an inflammatory condition, e.g., one or more clinical symptoms of IBD, for example one or more of the following symptoms:

a. Blood in the stool;

b. Elevated levels of fecal calprotectin;

c. Elevated levels of fecal lactoferrin;

d. Anemia;

e. Diarrhea;

f. Vomiting

g. Inappetence; or

h. Significant recent weight loss.

1.3.Any of Method 1, et seq. wherein the patient has failed to respond to antibiotics.
1.4.Any of Method 1, et seq. wherein said drug is selected from the group known to physicians consisting of aminosalicylates, corticosteroids, thiopurines, methotrexate, monoclonal antibodies, free bases thereof, pharmaceutically acceptable salts thereof, derivatives thereof, analogs thereof, and combinations thereof;

a. e.g., selected from one or more of
i. olsalazine
ii. mesalamine
iii. prednisone or prednisolone
iv. dexamethasone
v. budesonide (enteric coated)
vi. azathioprine
vii. cyclosporine.
   1.5.Any of Method 1, et seq. wherein the method further comprises assessing the patient's response to treatment by repeating the step of comprising detecting the presence and/or level of one or more inflammation-associated autoantibodies the patient in accordance with a method according to any one of Method A, et seq.
   1.6.Any of Method 1, et seq. further comprising the step of classifying the sample from the patient analyzed in accordance with Method 1, et seq., as being associated with a clinical subtype of IBD, said method comprising:

a. determining the presence or level of one or more markers selected from the group consisting of an anti-PMN antibody, anti-yeast antibody, antimicrobial antibody, calprotectin and combinations thereof in said sample; and

b. classifying said sample as a lymphoplasmacytic enteritis (LPE) sample, eosinophilic gastroenteritis (EGE) sample, granulomatous enteritis (GE) or non-IBD sample using a statistical algorithm based upon the presence or level of said one or more markers.

1.7.The preceding method wherein said statistical algorithm is selected from the group consisting of a classification and regression tree, boosted tree, neural network, random forest, support vector machine, general chi-squared automatic interaction detector model, interactive tree, multiadaptive regression spline, machine learning classifier, and combinations thereof.
1.8. Any of Method 1, et seq. further comprising giving the patient a diet with antigen-limited or hydrolyzed protein and/or high levels of insoluble fiber.

Other features and advantages of the invention are apparent from the following description of the embodiments thereof, and from the claims.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### EXAMPLES

The following examples are offered to illustrate, but not to limit, the claimed invention in any manner.

### Example 1

### Isolation of Canine Calprotectin Coding Regions and Preparation of Recombinant Polypeptides

This example illustrates the cloning of calprotectin coding regions and the preparation of calprotectin polypeptide fractions.

The coding regions of the calprotectin genes are cloned by assembling synthetic oligonucleotides. The synthetic constructs include NdeI and HindIII as flanking restriction sites on the 5′- and 3′-end of the gene of interest, respectively, and a histidine tag at the N-terminal region to create a HIS-calprotectin fusion polypeptide. The coding region sequences are designed to optimize polypeptide expression in E. coli. The assembled products are then subcloned into an expression vector with the N-terminal region of the coding gene operably linked to a start codon and an inducible promoter system. The expression constructs are transformed in E. coli BL21 and plated on LB agar plates containing kanamycin (50 μg/mL) for selection. Whole cell lysates are analyzed for clone selection. The amino-acid sequence of the genes are reported as SEQ ID NO: 1, SEQ ID NO: 2, SEQ ID NO: 3, and SEQ ID NO: 4 and correspond to nucleotide sequence of canine heterochimeric polypeptide S100A8/A9, canine polypeptide S100Al2, canine polypeptide S100A8, and canine polypeptide S100A9, respectively.

The following protocol describes the purification of a calprotectin polypeptide. The nucleic acid sequence of the calprotectin coding region is designed to include a polyhistidine tag to create a HIS-calprotectin fusion polypeptide. After expression in E. coli, the fusion polypeptide is purified using a nickel purification column. For inoculum preparation and for production, the recombinant E.coli cells are cultivated overnight (seed culture). The seed culture is then inoculated into a culture medium in larger flasks or mini-bioreactors at a ratio of 1 to 25 and cultured until reaching an optical density (OD) of 0.6-0.9 at 600 nm. At this cell density, cells are induced with 1 mM IPTG (Isopropyl β-D-1-thiogalactopyranoside) and the fermentation is carried out for another 4-16 hours. The cells are then harvested and lysed. The recombinant polypeptides are purified from the whole cell lysates using a nickel-charged purification resin. The purified recombinant polypeptides are shown to be of the expected molecular weight by Coomassie staining. Purified polypeptide preparations are diluted 5 times in a dimerization buffer (Dulbecco's Phosphate Buffered Saline (DPBS) with calcium, magnesium, 20% glycerol, 0.02% sodium azide, pH 7.0-7.2) and the reactions are incubated at 2-8° C. for at least 24 hours.

### Example 2

### Determination of Anti-Calprotectin Antibody (ACN) Levels in Dog Serum Samples

This example illustrates an analysis of anti-calprotectin antibody (ACN) levels in serum samples using a direct ELISA assay using various calprotectin polypeptides.

Detection of dog IgA antibodies that bind calprotectin (ACN-IgA) is performed by direct ELISA assays essentially as follows. ELISA plates are coated overnight at 4° C. with 100 l/well Calprotectin at 0.5 μg/mL in carbonate solution (100.0 mM NaHCO₃-Na₂CO₃ Buffer, pH 9.5±0.5). The plates are washed thrice with TBS-T (Tris Buffered Saline Tween, 25.0 mM Tris-HCl, 2.7 mM potassium chloride, 137 mM Sodium Chloride, 0.05% Tween-20, pH 7.4±0.2) and blocked with 200 μL/well TBS/BSA (Tris Buffered Saline, 25.0 mM Tris-HCl, 2.7 mM potassium chloride, 137 mM Sodium Chloride, pH 7.4±0.2, 1% BSA) for 1 hour at 18-25° C. After washing the plates thrice with TBS-T, the standard and sample preparations are added to each well and incubated at 18-25° C. for 1 hour. The plates are then washed thrice with TBS-T and incubated for 1 hour at 18-25° C. with horseradish peroxidase (HRP)-anti-dog IgA antibody diluted 1:5,000 in TBS/BSA. The plates are washed thrice with TBS-T and developed using 100 μL/well of 3,3′,5,5′-tetramethylbenzidine (TMB) substrate. The reaction is stopped with 0.33 M H₂SO₄ and the Optical Density (OD) is measured at 450 nm using an ELISA plate reader. The standard curve is fitted using a four parameter equation and used to estimate the antibody levels in the samples.

Typical results obtained with serum samples from diseased dogs and apparently healthy dogs (control) using the ELISA method described above are reported below. Data are compared using the Mann Whitney test and are expressed as Mean±Standard Error of the Mean (SEM) using Optical Density values. These results indicate that the calprotectin polypeptides derived from clones expressing SEQ ID NO:19, SEQ ID NO:20, SEQ ID NO:21, and SEQ ID NO:22 are differentially reactive with IBD sera as compared to normal sera and that the immunoreactivity to the calprotectin polypeptide, can be used to diagnose IBD.

TABLE 1
ACN-IgA levels in serum samples from diseased dogs and control dogs.

| Source of | | Diseased dogs | Control dogs | |
|---|---|---|---|---|
| Calprotectin | Description | Mean ± SEM | Mean ± SEM | p value |
| SEQ ID | Dimers of | 0.488 ± | 0.068 ± | 0.0027 |
| NO: 1 | heterochimeric | 0.126 | 0.017 | |
| | peptide | | | |
| | S100A8/S100A9 | | | |
| SEQ ID | Dimers of peptide | 0.539 ± | 0.062 ± | 0.0021 |
| NO: 2 | S100A12 | 0.138 | 0.017 | |
| SEQ ID | Dimers of peptide | 0.623 ± | 0.110 ± | 0.0027 |
| NO: 3 and | S100A8 and | 0.151 | 0.029 | |
| SEQ ID | peptide S100A9 | | | |
| NO: 4 | | | | |

### Example 3

### Determination of Anti-Calprotectin Antibody (ACN) Levels in Dog Serum Samples

This example illustrates an analysis of anti-calprotectin antibody (ACN) levels in a sample using a direct ELISA assay using the calprotectin polypeptide of SEQ ID NO: 1.

Detection of dog IgA antibodies that bind calprotectin (ACN-IgA) is performed by direct ELISA assays essentially as follows. ELISA plates are coated overnight at 4° C. with 100 μL/well Calprotectin at 0.5 μg/mL in carbonate solution (100.0 mM NaHCO3-Na2CO3 Buffer, pH 9.5±0.5). The plates are washed thrice with TBS-T (Tris Buffered Saline Tween, 25.0 mM Tris-HCl, 2.7 mM potassium chloride, 137 mM Sodium Chloride, 0.05% Tween-20, pH 7.4±0.2) and blocked with 200 μL/well TBS/BSA (Tris Buffered Saline, 25.0 mM Tris-HCl, 2.7 mM potassium chloride, 137 mM Sodium Chloride, pH 7.4±0.2, 1% BSA) for 1 hour at 18-25° C. After washing the plates thrice with TBS-T, the standard and sample preparations are added to each well and incubated at 18-25° C. for 1 hour. The plates are then washed thrice with TBS-T and incubated for 1 hour at 18-25° C. with horseradish peroxidase (HRP)-anti-dog IgA antibody diluted 1:5,000 in TBS/BSA. The plates are washed thrice with TBS-T and developed using 100 μL/well of 3,3′,5,5′-tetramethylbenzidine (TMB) substrate. The reaction is stopped with 0.33 M H2SO4 and the Optical Density (OD) is measured at 450 nm using an ELISA plate reader. The standard curve is fitted using a four parameter equation and used to estimate the antibody levels in the samples.

Typical results obtained with serum samples from diseased dogs (N=60) confirmed with the diagnosis of IBD by endoscopy followed by biopsy and apparently healthy dogs (controls, N=28) using the ELISA method described above are reported below. Data are compared using the Mann Whitney test and are expressed as Mean±Standard Error of the Mean (SEM) using EU (Elisa Units). These results indicate that the calprotectin polypeptide derived from clones expressing SEQ ID NO:1 is differentially reactive with IBD sera as compared to normal sera and that the immunoreactivity to the calprotectin polypeptide, can be used to diagnose IBD.

TABLE 2
ACN-IgA levels in serum samples from diseased dogs and control dogs.

| | | Diseased dogs | Control dogs | |
|---|---|---|---|---|
| Source of | | Mean ± SEM | Mean ± SEM | |
| Calprotectin | Description | (EU) | (EU) | p value |
| SEQ ID | Dimers of | 45.45 ± | 3.849 ± | <0.0001 |
| NO: 19 | heterochimeric | 12.71 | 0.488 | |
| | peptide | | | |
| | S100A8/S100A9 | | | |

### Example 4

### Isolation of Canine Integrin Coding Regions and Preparation of Recombinant Polypeptides

This example illustrates the cloning of integrin coding regions and the preparation of integrin polypeptide fractions.

Fragments of the coding regions of canine integrin alpha-4 and canine integrin beta-7 are cloned by PCR amplification using cDNA isolated from dog as template. PCR reactions are carried out in a 25 μL final volume containing the reaction master mix supplemented with a Taq DNA polymerase (Thermo Fisher scientific), the DNA template, and 0.5 μM of each of a forward primer and of reverse primer. For amplification of fragments of the integrin alpha-4 coding region, forward primers of SEQ ID NO:23 and SEQ ID NO:24 and reverse primer of SEQ ID NO:25 are used. For amplification of fragments of the integrin beta-7 coding region, forward primer of SEQ ID NO:26 and reverse primers of SEQ ID NO:27 and SEQ ID NO:28 are used. The PCR reaction mix is denatured at 94° C. for 4-5 min followed by amplification for 30 cycles (95° C. for 30 s, 50° C. for 30 s, 72° C. for 60 s) and an extension at 72° C. for 10 min. The amino-acid sequence of the cloned fragments of the integrin alpha-4 coding region are reported as SEQ ID NO:29 and SEQ ID NO:30. The amino-acid sequence of the cloned fragments of the integrin beta-7 coding region are reported as SEQ ID NO:31 and SEQ ID NO:32. The PCR products are cloned into a bacterial expression vector containing a histidine tag according to the manufacturer's recommendations (Life Technologies).

The following protocol describes the preparation of purified recombinant integrin polypeptides. The nucleic acid sequence of the integrin coding region includes a polyhistidine tag to create a HIS-Integrin fusion polypeptide. After expression in E. coli, the fusion polypeptide is purified using a nickel purification column. For inoculum preparation and for production, the recombinant E.coli cells are cultivated overnight (seed culture). The seed culture is inoculated into culture medium in larger flasks or mini-bioreactors at a ratio of 1 to 25 and cultured until reaching an optical density (OD) of 0.6-0.9 at 600 nm. At this cell density, cells are induced with 1mM IPTG (Isopropyl β-D-1-thiogalactopyranoside) and the fermentation is carried out for another 4-16 hours. The cells are then harvested and lysed. The recombinant polypeptides are purified from the whole cell lysates using a nickel-charged purification resin. The purified recombinant polypeptides are shown to be of the expected molecular weight by Coomassie staining.

### Example 5

### Determination of Anti-Integrin Antibody (AIN) Levels in Dog Serum Samples

This example illustrates an analysis of anti-integrin antibody (AIN) levels in a sample using a direct ELISA assay.

Detection of dog IgA antibodies that bind integrin (AIN-IgA) is performed by direct ELISA assays essentially as follows. ELISA plates are coated overnight at 4° C. with 100 μL/well with the integrin polypeptide preparation at 0.2 μg/mL in carbonate solution (100.0 mM NaHCO₃-Na₂CO₃ Buffer, pH 9.5±0.5). The plates are washed thrice with TBS-T (Tris Buffered Saline Tween, 25.0 mM Tris-HCl, 2.7 mM potassium chloride, 137 mM Sodium Chloride, 0.05% Tween-20, pH 7.4±0.2) and blocked with 200 μL/well TBS/BSA (Tris Buffered Saline, 25.0 mM Tris-HCl, 2.7 mM potassium chloride, 137 mM Sodium Chloride, pH 7.4±0.2, 1% BSA) for 1 hour at 18-25° C. After washing the plates thrice with TBS-T, the standard and sample preparations are added to each well and incubated at 18-25° C. for 1 hour. The plates are then washed thrice with TBS-T and incubated for 1 hour at 18-25° C. with horseradish peroxidase (HRP)-anti-dog IgA antibody diluted 1:5,000 in TBS/BSA. The plates are washed thrice with TBS-T and developed using 100 μL/well of 3,3′,5,5′-tetramethylbenzidine (TMB) substrate. The reaction is stopped with 0.33 M H₂SO₄ and the Optical Density (OD) is measured at 450 nm using an ELISA plate reader. The standard curve is fitted using a four parameter equation and used to estimate the antibody levels in the samples.

Typical results obtained with serum samples from diseased dogs and apparently healthy dogs (control) using the ELISA method described above are reported below. Data are compared using the Mann Whitney test and are expressed as Mean±Standard Error of the Mean (SEM) using EU (Elisa Units). In addition, area under the curve (AUC) from receiver operating characteristics (ROC) curves generated by plotting sensitivity versus 1□specificity for each integrin polypeptide are shown.

These results indicate that the integrin polypeptide preparations derived from clones expressing SEQ ID NO: 11, SEQ ID NO: 12, SEQ ID NO: 13, and SEQ ID NO: 14 are differentially reactive with IBD sera as compared to normal sera and that the immunoreactivity to the integrin polypeptide, can be used to diagnose IBD.

TABLE 3
AIN-IgA levels in serum samples from diseased dogs and control dogs.

| | | Diseased Dogs | Control Dogs | |
|---|---|---|---|---|
| | Integrin | Mean ± SEM | Mean ± SEM | |
| Integrin | Polypeptides | (EU) | (EU) | p value |
| SEQ ID NO: 11 | α4 | 168.8 ± 55.74 | 16.92 ± 6.49 | 0.0001 |
| SEQ ID NO: 12 | α4 | 149.1 ± 54.65 | 24.78 ± 5.81 | 0.002 |
| SEQ ID NO: 13 | β7 | 149.3 ± 51.56 | 16.06 ± 4.12 | 0.0002 |
| SEQ ID NO: 14 | β7 | 145.9 ± 48.17 | 26.43 ± 6.26 | 0.0057 |
| SEQ ID NO: 11 & | α4 & β7 | 165.2 ± 55.95 | 23.73 ± 6.43 | 0.0013 |
| SEQ ID NO: 13 | | | | |

TABLE 4
Area under the curve values (AUC) obtained for ROC curves using
different integrin polypeptides for differentiation between control dogs
and diseased dogs.

| | AUC | Std. Error | P value |
|---|---|---|---|
| SEQ ID NO: 11 | 0.844 | 0.069 | 0.0005 |
| SEQ ID NO: 12 | 0.784 | 0.079 | 0.0038 |
| SEQ ID NO: 13 | 0.850 | 0.062 | 0.0004 |
| SEQ ID NO: 14 | 0.750 | 0.088 | 0.0109 |
| SEQ ID NO: 11 and | 0.797 | 0.079 | 0.0025 |
| SEQ ID NO: 13 | | | |

### Example 6

### Determination of Anti-Calprotectin Antibody IgA (ACN-IgA) Levels in Human Serum Samples

This example illustrates an analysis of anti-calprotectin antibody IgA (ACN) levels in human serum samples using a direct ELISA assay.

Detection of human IgA antibodies that bind calprotectin (ACN-IgA) is performed by direct ELISA assays essentially as follows using human serum from apparently normal (N) and Inflammatory Bowel Disease (IBD) subjects, in particular Ulcerative Colitis (UC), and Crohn's Disease (CD) subjects.

ELISA plates are coated overnight at 4° C. with 100 μL/well with a recombinant, E. coli derived, human calprotectin S100A8/S100A9 heterodimer (R&D Systems, Cat No. 8226-S8) at 0.2 m/mL in carbonate solution (100.0 mM NaHCO₃-Na₂CO₃ Buffer, pH 9.5±0.5). The plates are washed thrice with TBS-T (Tris Buffered Saline Tween, 25.0 mM Tris-HCl, 2.7 mM potassium chloride, 137 mM Sodium Chloride, 0.05% Tween-20, pH 7.4±0.2) and blocked with 200 μL/well TBS/BSA (Tris Buffered Saline, 25.0 mM Tris-HCl, 2.7 mM potassium chloride, 137 mM Sodium Chloride, pH 7.4±0.2, 1% BSA) for 1 hour at 18-25° C. After washing the plates thrice with TBS-T, the standard and sample preparations are added to each well and incubated at 18-25° C. for 1 hour. The plates are then washed thrice with TBS-T and incubated for 1 hour at 18-25° C. with horseradish peroxidase (HRP)-anti-human IgA antibody diluted 1:2,000 in TBS/BSA. The plates are washed thrice with TBS-T and developed using 100 μL/well of 3,3′,5,5′-tetramethylbenzidine (TMB) substrate. The reaction is stopped with 0.33 M H₂SO₄ and the Optical Density (OD) is measured at 450 nm using an ELISA plate reader.

Results obtained using the ELISA method described above with human serum samples from IBD subjects, in particular Ulcerative Colitis (UC) and Crohn's Disease (CD) subjects and apparently normal subjects are reported below. Data are compared using the Mann Whitney test and are expressed as Mean±Standard Error of the Mean (SEM) using Optical Density values. These results indicate that the calprotectin is differentially reactive with IBD sera as compared to normal sera and that the immunoreactivity to the calprotectin polypeptide, can be used to diagnose IBD.

TABLE 5
ACN-IgA levels in human serum samples from control subjects (normal)
and diseased subjects (Ulcerative Colitis and Crohn's disease)

| Subject Groups | Description | Mean ± SEM |
|---|---|---|
| Group 1 | Ulcerative Colitis (UC) | 0.527 ± 0.052 |
| Group 2 | Apparently Normal (N) | 0.442 ± 0.023 |
| Group 3 | Crohn's Disease (CD) | 0.779 ± 0.068 |
| Mann Whitney Test | | P value |
| Group 1 vs Group 2 | UC vs N | 0.17 |
| Group 2 vs Group 3 | N vs CD | <0.0001 |

### Example 7

### Determination of Anti-Calprotectin Antibody IgG (ACN-IgG) Levels in Human Serum Samples

This example illustrates an analysis of anti-calprotectin antibody IgG (ACN-IgG) levels in human serum samples using a direct ELISA assay.

Detection of human IgG antibodies that bind calprotectin (ACN-IgG) is performed by direct ELISA assays essentially as follows using human serum from apparently normal (N) and Inflammatory Bowel Disease (IBD) subjects, in particular Ulcerative Colitis (UC), and Crohn's Disease (CD) subjects.

ELISA plates are coated overnight at 4° C. with 100 μL/well with a recombinant, E. coli derived, human calprotectin S100A8/S100 A9 heterodimer (R&D Systems, Cat No. 8226-S8) at 0.2m/mL in carbonate solution (100.0 mM NaHCO₃-Na₂CO₃ Buffer, pH 9.5±0.5). The plates are washed thrice with TBS-T (Tris Buffered Saline Tween, 25.0 mM Tris-HCl, 2.7 mM potassium chloride, 137 mM Sodium Chloride, 0.05% Tween-20, pH 7.4±0.2) and blocked with 200 μL/well TBS/BSA (Tris Buffered Saline, 25.0 mM Tris-HCl, 2.7 mM potassium chloride, 137 mM Sodium Chloride, pH 7.4±0.2, 1% BSA) for 1 hour at 18-25° C. After washing the plates thrice with TBS-T, the standard and sample preparations are added to each well and incubated at 18-25° C. for 1 hour. The plates are then washed thrice with TBS-T and incubated for 1 hour at 18-25° C. with horseradish peroxidase (HRP)-anti-human IgG antibody diluted 1:10,000 in TBS/BSA. The plates are washed thrice with TBS-T and developed using 100 μL/well of 3,3′,5,5′-tetramethylbenzidine (TMB) substrate. The reaction is stopped with 0.33 M H₂SO₄ and the Optical Density (OD) is measured at 450 nm using an ELISA plate reader.

Results obtained using the ELISA method described above with human serum samples from IBD subjects, in particular Ulcerative Colitis (UC) and Crohn's Disease (CD) subjects and apparently normal subjects are reported below. Data are compared using the Mann Whitney test and are expressed as Mean±Standard Error of the Mean (SEM) using Optical Density values.

TABLE 6
ACN-IgG levels in human serum samples from control subjects (normal)
and diseased subjects (Ulcerative Colitis and Crohn's disease)

| Subject Groups | Description | Mean ± SEM |
|---|---|---|
| Group 1 | Ulcerative Colitis (UC) | 0.584 ± 0.078 |
| Group 2 | Apparently Normal (N) | 0.510 ± 0.048 |
| Group 3 | Crohn's Disease (CD) | 0.639 ± 0.076 |
| Mann Whitney Test | | P value |
| Group 1 vs Group 2 | UC vs N | 0.2949 |
| Group 2 vs Group 3 | N vs CD | 0.3093 |

### Example 8

### Determination of Anti-Integrin Antibody IgA (AIN-IgA) Levels in Human Serum Samples

This example illustrates an analysis of anti-integrin antibody IgA (AIN-IgA) levels in human serum samples using a direct ELISA assay.

Detection of human IgA antibodies that bind integrin (AIN-IgA) is performed by direct ELISA assays essentially as follows using human serum from apparently normal (N) and Inflammatory Bowel Disease (IBD) subjects, in particular Ulcerative Colitis (UC), and Crohn's Disease (CD) subjects.

ELISA plates are coated overnight at 4° C. with 100 μL/well with a recombinant, CHO cell derived, human Integrin alpha-4 beta-7 (R&D Systems, Cat No. 5397-A3) at 0.2 μg/mL in carbonate solution (100.0 mM NaHCO₃-Na₂CO₃ Buffer, pH 9.5±0.5). The plates are washed thrice with TBS-T (Tris Buffered Saline Tween, 25.0 mM Tris-HCl, 2.7 mM potassium chloride, 137 mM Sodium Chloride, 0.05% Tween-20, pH 7.4±0.2) and blocked with 200 μL/well TBS/BSA (Tris Buffered Saline, 25.0 mM Tris-HCl, 2.7 mM potassium chloride, 137 mM Sodium Chloride, pH 7.4±0.2, 1% BSA) for 1 hour at 18-25° C. After washing the plates thrice with TBS-T, the standard and sample preparations are added to each well and incubated at 18-25° C. for 1 hour. The plates are then washed thrice with TBS-T and incubated for 1 hour at 18-25° C. with horseradish peroxidase (HRP)-anti-human IgA antibody diluted 1:2,000 in TBS/BSA. The plates are washed thrice with TBS-T and developed using 100 μL/well of 3,3′,5,5′-tetramethylbenzidine (TMB) substrate. The reaction is stopped with 0.33 M H₂SO₄ and the Optical Density (OD) is measured at 450 nm using an ELISA plate reader.

Results obtained using the ELISA method described above with human serum samples from IBD subjects, in particular Ulcerative Colitis (UC) and Crohn's Disease (CD) subjects and apparently normal subjects are reported below. Data are compared using the Mann Whitney test and are expressed as Mean±Standard Error of the Mean (SEM) using Optical Density values. These results indicate that the integrin alpha-4 beta-7 is differentially reactive with IBD sera as compared to normal sera and that the immunoreactivity to the integrin polypeptide, can be used to diagnose IBD.

TABLE 7
AIN-IgA levels in human serum samples from control subjects (normal)
and diseased subjects (Ulcerative Colitis and Crohn's disease)

| Subject Groups | Description | Mean ± SEM |
|---|---|---|
| Group 1 | Ulcerative Colitis (UC) | 0.485 ± 0.043 |
| Group 2 | Apparently Normal (N) | 0.387 ± 0.024 |
| Group 3 | Crohn's Disease (CD) | 0.695 ± 0.057 |
| Mann Whitney Test | | P value |
| Group 1 vs Group 2 | UC vs N | 0.065 |
| Group 2 vs Group 3 | N vs CD | <0.0001 |

### Example 9

### Determination of Anti-Integrin Antibody IgG (AIN-IgG) Levels in Human Serum Samples

This example illustrates an analysis of anti-integrin antibody IgG (AIN-IgG) levels in human serum samples using a direct ELISA assay.

Detection of human IgG antibodies that bind integrin (AIN-IgG) is performed by direct ELISA assays essentially as follows using human serum from apparently normal (N) and Inflammatory Bowel Disease (IBD) subjects, in particular Ulcerative Colitis (UC), and Crohn's Disease (CD) subjects.

ELISA plates are coated overnight at 4° C. with 100 μL/well with a recombinant, CHO cell derived, human Integrin alpha-4 beta-7 (R&D Systems, Cat No. 5397-A3) at 0.2 μg/mL in carbonate solution (100.0 mM NaHCO₃-Na₂CO₃ Buffer, pH 9.5±0.5). The plates are washed thrice with TBS-T (Tris Buffered Saline Tween, 25.0 mM Tris-HCl, 2.7 mM potassium chloride, 137 mM Sodium Chloride, 0.05% Tween-20, pH 7.4±0.2) and blocked with 200 μL/well TBS/BSA (Tris Buffered Saline, 25.0 mM Tris-HCl, 2.7 mM potassium chloride, 137 mM Sodium Chloride, pH 7.4±0.2, 1% BSA) for 1 hour at 18-25° C. After washing the plates thrice with TBS-T, the standard and sample preparations are added to each well and incubated at 18-25° C. for 1 hour. The plates are then washed thrice with TBS-T and incubated for 1 hour at 18-25° C. with horseradish peroxidase (HRP)-anti-human IgG antibody diluted 1:10,000 in TBS/BSA. The plates are washed thrice with TBS-T and developed using 100 μL/well of 3,3′,5,5′-tetramethylbenzidine (TMB) substrate. The reaction is stopped with 0.33 M H₂SO₄ and the Optical Density (OD) is measured at 450 nm using an ELISA plate reader.

Results obtained using the ELISA method described above with human serum samples from IBD subjects, in particular Ulcerative Colitis (UC) and Crohn's Disease (CD) subjects and apparently normal subjects are reported below. Data are compared using the Mann Whitney test and are expressed as Mean±Standard Error of the Mean (SEM) using Optical Density values.

TABLE 8
AIN-IgG levels in human serum samples from control subjects (normal)
and diseased subjects (Ulcerative Colitis and Crohn's disease)

| Subject Groups | Description | Mean ± SEM |
|---|---|---|
| Group 1 | Ulcerative Colitis (UC) | 0.477 ± 0.057 |
| Group 2 | Apparently Normal (N) | 0.510 ± 0.050 |
| Group 3 | Crohn's Disease (CD) | 0.596 ± 0.072 |
| Mann Whitney Test | | P value |
| Group 1 vs Group 2 | UC vs N | 0.2034 |
| Group 2 vs Group 3 | N vs CD | 0.1186 |

### Example 10

### Determination of ACA, APMNA, ACNA, and AFA Levels in Dog Serum Samples

This example illustrates an analysis of anti-OmpC antibody level (ACA), anti-canine polymorphonuclear leukocytes antibody level (APMNA), anti-calprotectin antibody level (ACNA), and anti-flagellin antibody level (AFA) using a direct ELISA assay in serum samples. Serum samples are collected from three cohorts of dogs: (i) the “IBD Dog” cohort includes dogs confirmed with the diagnosis of IBD based on the chronicity of gastrointestinal signs, the exclusion of underlying infectious, endocrine or neoplastic diseases, and the histological inflammatory findings; (ii) the “Non-IBD” cohort includes dogs predominantly with acute gastrointestinal symptoms; and (iii) the “Normal Dog” cohort includes dogs with no apparent gastrointestinal symptoms.

### Study Design and Inclusion Criteria.

This is a multicenter study designed to develop methods and systems to accurately detect and measure the presence and/or levels of endogenous antibodies to markers associated with inflammatory bowel disease (IBD) in dogs. Such methods and systems identify whether a sample from the patient is associated with an inflammatory condition, by using non-invasive means, thus conveniently providing information useful for guiding treatment decisions. In this study, serum samples are collected once from dogs of the IBD cohort with gastrointestinal symptoms and from dogs of the Normal cohort with no apparent gastrointestinal symptoms. Dog owners sign an informed consent form for their dogs to participate in the study. IBD Dogs are considered eligible for participation if they meet the following inclusion criteria: vomiting, diarrhea, anorexia, weight loss, or some combination of these signs for at least 3 weeks; no immunosuppresive drugs or antibiotics administered for at least 10 days before sample collection; and confirmation of IBD by histopathology analysis of biopsy samples. Dogs are confirmed with the diagnosis of IBD based on the chronicity of gastrointestinal signs, the exclusion of underlying infectious, endocrine or neoplastic diseases, and the histological inflammatory findings. A complete clinical evaluation is performed, including hematology, clinical biochemistry, and as required, fecal flotation, Giardia antigen test, and abdominal ultrasound to exclude infectious, endocrine or neoplastic diseases. Gastroduodenoscopy is performed in all dogs of the IBD cohort, and biopsy samples from the stomach, duodenum, and colon, are collected with flexible endoscopy biopsy forceps. All IBD dogs are scored according to the canine inflammatory bowel disease activity index (CIBDAI). Full thickness biopsies and/or endoscopy biopsies are immediately placed in ice-cold phosphate-buffered saline (PBS) and 4% buffered paraformaldehyde solution until processed. All tissue samples are processed and graded by a clinical pathologist according using the World Small Animal Veterinary Association (WSAVA) guidelines. Multiple morphological parameters (i.e. epithelial injury, crypt distension, lacteal dilatation, mucosal fibrosis) and inflammatory histological parameters (such as plasma cells, lamina propria lymphocyte, eosinophils and neutrophils) are scored, and the resulting final scores are subdivided into histological severity groups: WSAVA score of 0=normal, 1-6=mild, 7-12=moderate, >13=severe.

### Determination of Antibody Levels in Dog Sera to OMPC, PMN, Calprotectin, and Flagellin.

Canine IgA antibody levels against specific antigens are detected by direct ELISA assays. Sera from the IBD Dog, Non-IBD Dog, and Normal Dog cohorts are analyzed in duplicate for IgA reactivity to OmpC (ACA-IgA), canine polymorphonuclear leukocytes (APMNA-IgA), canine calprotectin (ACNA-IgA), and flagellin (AFA-IgA) as described previously.

The recombinant polypeptides for OmpC, calprotectin, and flagellin, utilized for the preparation of the coating material are peptides of sequences SEQ ID No: 17, SEQ ID No: 1, and SEQ ID No: 16, respectively. PMNs are isolated from canine blood as described in Example 2.

Briefly, for determination of APMNA-IgA levels in serum, microtiter plates are coated with 12.5×10³ to 200×10³ PMN per well isolated from blood sample collected from a single dog. A layer of PMN is recovered after centrifugation of the whole blood at 18-25° C. and treated with a hypotonic solution to lyse red blood cells. PMN are treated with cold 95% methanol and 5% acetic acid for 20±10 minutes to fix the cells. Cells are incubated for 60±30 minutes at 18-25° C. with 1% bovine serum albumin (BSA) in phosphate-buffered saline to block nonspecific antibody binding. Next, after 3 washes with Tris Buffered Saline-Tween (TBS-T: Tris Buffered Saline Tween, 25.0 mM Tris-HCl, 2.7 mM potassium chloride, 137 mM Sodium Chloride, 0.05% Tween-20, pH 7.4±0.2), control sera and test sample sera are added at a 1:50 to 1:100 dilutions to the microtiter plates and incubated for 60±30 minutes at 18-25° C. After 3 washes with TBS-T, alkaline phosphatase-conjugated anti-dog IgA is added at a 1:2000 dilution to label PMN-bound antibody and incubated for 60±30 minutes at 18-25° C. A solution of p-nitrophenol phosphate substrate is added, and color development is allowed to proceed for 30±10 minutes. The Optical Density (OD) is measured at 405 nm using an ELISA plate reader.

For all other markers, microtiter plates are coated overnight at 4° C. with 100 μL/well at 0.2 μg/mL to 0.5m/mL antigen in carbonate solution (100.0 mM NaHCO₃-Na₂CO₃ Buffer, pH 9.5±0.5). The plates are washed thrice with TBS-T (Tris Buffered Saline Tween, 25.0 mM Tris-HCl, 2.7 mM potassium chloride, 137 mM Sodium Chloride, 0.05% Tween-20, pH 7.4±0.2) and blocked with 200 μL/well TBS/BSA (Tris Buffered Saline, 25.0 mM Tris-HCl, 2.7 mM potassium chloride, 137 mM Sodium Chloride, pH 7.4±0.2, 1% BSA) for 1 hour at 18-25° C. After washing the plates thrice with TBS-T, the standard and sample preparations are added to each well and incubated at 18-25° C. for 1 hour. The plates are then washed thrice with TBS-T and incubated for 1 hour at 18-25° C. with horseradish peroxidase (HRP)-anti-dog IgA antibody diluted 1:5,000 in TBS/BSA. The plates are washed thrice with TBS-T and developed using 100 μL/well of 3,3′,5,5′-Tetramethylbenzidine (TMB) substrate. The reaction is stopped with 0.33 M H₂SO₄ and the Optical Density (OD) is measured at 450 nm using an ELISA plate reader.

Antibody levels are determined relative to a standard/calibrator/reference obtained from a dog with a positive signal using the Softmax software (Molecular Devices). Results with test samples are expressed as ELISA units/mL. Sera with circulating ACA, APMNA, ACNA, and AFA levels greater than two standard deviations above the mean value of the normal cohort may respectively be termed ACA positive, APMNA positive, ACNA positive, and AFA positive whereas numerical values that are less than the reference values may be termed negative.

### Statistical Analysis

Statistical analysis is conducted using the Graphpad Prism (GraphPad Software, La Jolla Calif. USA) or Microsoft Office Excel (2013, Microsoft, Redmond, Wash., USA). Mean, median, minimum, maximum, and percentile are calculated. Data are analyzed by ANOVA with Bonferroni's post hoc multiple comparison test and presented as the mean (±SEM) and p values. Statistical analyses include area under receiver operating characteristic (ROC) curves and calculations of diagnostic sensitivity and specificity as appropriate for each of the markers (univariate analysis) and for a combination of markers (multivariate analysis). Measures of performance, sensitivity and specificity, may be computed using multiple reference values. A p-value <0.05 is considered significant.

### Results.

The IBD-Dog cohort includes seventy dogs of various ages, gender and breeds presenting with chronic gastrointestinal signs. The Non-IBD-Dog cohort includes twenty-three dogs predominantly presenting with acute gastrointestinal symptoms. The Normal-Dog cohort consists of fifty eight dogs of various ages, gender, and breeds presenting no significant gastrointestinal symptoms at the time of visit at the clinical site.

Levels of IgA antibodies to OmpC (ACA), canine polymorphonuclear leukocytes (APMNA), calprotectin (ACNA), and flagellin (AFA) are determined in all enrolled subjects.

Typical results obtained with serum samples from IBD-Dogs and Normal-Dogs using the ELISA method described above are reported below. Data are compared between groups using the area under the curve (AUC) from receiver operating characteristics (ROC) curves generated by plotting sensitivity versus 1-specificity for each marker. These results indicate that the markers are differentially reactive with IBD-Dog sera as compared to Normal-Dog sera and Non-IBD-Dog sera, and that the immunoreactivity to the markers can be used to detect IBD.

TABLE 9
Area under the curve values (AUC) obtained for ROC curves using
OmpC (ACA), PMN (APMNA), calprotectin (ACNA), and flagellin
(AFA) markers for differentiation between the IBD Dog and Normal
Dog cohorts.

| | ACA-IgA | APMNA-IgA | ACNA-IgA | AFA-IgA |
|---|---|---|---|---|
| Area under the | 0.915 | 0.924 | 0.774 | 0.766 |
| ROC curve | | | | |
| P value | <0.0001 | <0.0001 | <0.0001 | <0.0001 |
| Specificity | 93% | 91% | 86% | 80% |
| Sensitivity | 87% | 86% | 66% | 64% |
| Indeterminate | 4% | 10% | 7% | 21% |

The table below summarizes the percent of positive samples identified in the IBD, Non-IBD, and Normal cohort. Samples with values greater than two standard deviations above the mean value of the normal cohort are identified as positive samples. The data show that the number of positive samples is significantly higher in the IBD cohorts.

TABLE 10
Percentage of positive serum samples per cohort.

| Cohort | ACA-IgA | APMNA-IgA | ACNA-IgA | AFA-IgA |
|---|---|---|---|---|
| IBD-Dogs | 75.7 | 77.1 | 42.9 | 38.6 |
| Non-IBD Dogs | 13.0 | 13.0 | 13.0 | 0.0 |
| Normal Dogs | 3.4 | 8.6 | 8.6 | 8.6 |

Data are analyzed by ANOVA with Bonferroni's post hoc multiple comparison test and the p value and the mean (±SEM) are is presented in the table below. The data show that there is a significant statistical difference between the IBD Dog vs the Non-IBD Dog cohorts and IBD Dog vs the Normal Dog cohorts. There is no significant statistical difference between the Normal Dog vs Non-IBD Dog cohorts.

TABLE 11
P values results obtained for four markers, ACA, APMNA, ACNA,
and AFA, by ANOVA analysis with Bonferroni's post hoc multiple
comparison test.

| Cohort Comparison | ACA | APMNA | ACNA | AFA |
|---|---|---|---|---|
| Normal vs IBD | <0.0001 | 0.0005 | 0.0009 | <0.0001 |
| Non-IBD vs IBD | <0.0001 | <0.0001 | 0.0166 | <0.0001 |
| Normal vs Non-IBD | 0.6231 | 0.7873 | 0.9051 | 0.7770 |

TABLE 12
Mean ± SEM results obtained for four markers, ACA, APMNA, ACNA,
and AFA for the IBD Dog, Non-IBD Dog, and Normal Dog cohorts.

| Cohort | IBD | Non-IBD | Normal |
|---|---|---|---|
| ACA | 251.5 ± 29.40 | 31.51 ± 18.48 | 10.15 ± 1.96 |
| APMNA | 121.8 ± 12.42 | 26.04 ± 5.15 | 20.96 ± 1.42 |
| ACNA | 47.22 ± 11.04 | 9.072 ± 1.50 | 6.852 ± 0.68 |
| AFA | 189.7 ± 31.82 | 13.5 ± 3.11 | 26.66 ± 5.14 |

Overall, these results indicate that the method of detecting in a sample the presence and/or level of endogenous antibodies to OmpC, canine polymorphonuclear leukocytes, calprotectin, and flagellin, markers associated with inflammatory bowel disease (IBD), can be utilized to evaluate IBD in dogs.

### Example 17

### Determination of ACA and ACNA in Dog Serum Samples in a Longitudinal Study

This example illustrates an analysis of anti-OmpC antibody level (ACA) and anti-calprotectin antibody level (ACNA) using dog serum samples to monitor the marker levels during the evolution of the disease.

In this study, serum samples are collected from dogs with gastrointestinal symptoms such as vomiting, diarrhea, anorexia, weight loss, or some combination for a long period of time. Serum samples are collected at the initial visit and may be collected as a follow-up visit after completion of treatment prescribed by the attending clinician.

Serum samples are collected and stored for short period of time at 2 to 8° C. and for long period of time at −10 to −20° C. until analysis.

Levels of canine IgA antibodies to OmpC (ACA) and calprotectin (ACNA) are determined using a direct ELISA method described previously.

Antibody levels are determined relative to a standard/calibrator/reference obtained from a dog with a positive signal using the Softmax software (Molecular Devices). Results with test samples are expressed as ELISA units/mL. Sera with circulating ACA and ACNA levels may be categorized as low, intermediate, or high. These three categories are defined by analysis of area under receiver operating characteristic (ROC) curves and calculations of diagnostic sensitivity and specificity as appropriate for each of the markers (univariate analysis) and for a combination of markers (multivariate analysis).

Typical results are listed below for dogs categorized as positive by testing for immunoreactivity to OmpC and calprotectin.

TABLE 13
ACA-IgA and ACNA-IgA level results obtained by using a direct
ELISA method fromserum samples collected from dogs with
gastrointestinal symptoms.

| Subject | Serum Samples | ACA-IgA (EU/mL) | ACNA-IgA (EU/mL) |
|---|---|---|---|
| Dog 1 | Initial Visit | 2,021.6 (High) | 60.5 (High) |
| Dog 1 | Follow-up Visit | 497.6 (High) | 60.1 (High) |
| Dog 2 | Initial Visit | 42.4 (High) | 9.5 (Intermediate) |
| Dog 2 | Follow-up Visit | 2.7 (Low) | 4.9 (Low) |

Evidence of inflammatory bowel disease is confirmed by a pathologist based on a biopsy performed on the dog tested for seropositivity for OmpC and calprotectin. For instance, moderate lymphomplasmacytic enteritis with eosinophils and mild lymphoplasmacytic gastritis is observed for dog 2: sections of tissue from the stomach are characterized by mild inflammation with a mild accumulation of lymphocytes and plasma cells within the gastric mass; sections of tissue from the intestine are characterized by a moderate inflammation with a moderate accumulation of lymphocytes and plasma cells within the lamina propria, villous structures are swollen and lacteals are occasionally dilated at the villous tips.

These results indicate that the method of detecting the presence and/or level of one or more endogenous antibodies associated with inflammatory bowel disease (IBD) in a sample can be utilized to detect and monitor IBD.

SEQUENCE LISTING

| SEQ ID NO | Gene | Sequence |
|---|---|---|
| SEQ ID NO: 1 | Hetero-chimeric | MGSSHHHHHHGLTELESAINSLIEVYHKYSLVKGNYHALYRDD |
| | S100A8/S100A9 | LKKLLETECPQYMKKKDADTWFQELDVNSDGAINFEEFLILVI |
| | | KVGVASHKDIHKEGGGGSGGGGSGGGGSADQMSQLECSIETII |
| | | NIFHQYSVRLEHPDKLNQKEMKQLVKKELPNFLKKQKKNDNAI |
| | | NKIMEDLDTNGDKELNFEEEFSILVARLTVASHEEMHKNAPEG |
| | | EGHSHGPGFGEGSQGHCHSHGGHGHGHSH |
| SEQ ID NO: 2 | S100A12 | MGSSHHHHHHGTKLEDHLEGIVDVFHRYSARVGHPDTLSKGEMK |
| | | QLIIRELPNTLKNTKDQATVDKLFQDLDADKDGQVNFNEFISLV |
| | | SVVLDTSHKNTHKE |
| SEQ ID NO: 3 | S100A8 | MGSSHHHHHHGLTELESAINSLIEVYHKYSLVKGNYHALRDDLK |
| | | KLLETECPQYMKKKDADTWFQELDVNSDGAINFEEFLILVIKVG |
| | | VASHKDIHKE |
| SEQ ID NO: 4 | S100A9 | MGSSHHHHHHGADQMSQLECSIETIINIFHQYSVRLEHPDKLNQ |
| | | KEMKQLVKKELPNFLKKQKKNDNAINKIMEDLDTNGDKELNFEE |
| | | FSILVARLTVASHEEMHKNAPGEGEGHSHGPGFGEGSQGFFIXH |
| | | GGHGHGHSH |
| SEQ ID NO: 5 | α4 | 5′-GTGTCTGCCTCTCGACCTCGG-3′ |
| SEQ ID NO: 6 | α4 | 5′-CAGAGAATTGAAGGATTTCAAATCAGC-3′ |
| SEQ ID NO: 7 | α4 | REV: 5′-TTATGTGAAATGACGTTTGGGTCTTTG-3′ |
| SEQ ID NO: 8 | β7 | FW: 5′-GAATTGGATGCCAAGATCTCC-3′ |
| SEQ ID NO: 9 | β7 | REV: 5′-TTACAGTGTGTGCAGCTCCACAGTCAG-3′ |
| SEQ ID NO: 10 | β7 | REV: 5′-TTAGTGATCCGCGCCTCTCTCTTG-3′ |
| SEQ ID NO: 11 | α4 | WLVVGAPTARWLANSAVVNPGAIYRCRIGGNPGLTCEQLQLGSP |
| | | SGEPCKTCLEERDNQWLGVTLSRQPGENGSIVTCGHRWKNIFYI |
| | | KNENKLPMGVCYGMPSDLRTELSKRIAPCYQDYVRKFGENFASC |
| | | QAGISSFYTEDLIVMGAPGSSYWTGSLFVYNITTNKYKAFLDRQ |
| | | NQVKFGSYLGYSVGAGHFRSPHTTEVVGGAPQHEQIGKAYIFSI |
| | | EAKELSILHEMKGKKLGSYFGASVCAVDLNADGFSDLLVGAPMQ |
| | | STIREEGRVFVYINSGSGAVMNEMETELIGSDKYAARFGESIVN |
| | | LGDIDNDGFEDVAVGAPQEDDLRGAVYIYNGRADGISTAFSQRI |
| | | EGFQISKSLSMFGQSISGQIDADNNGYVDVAVGAFRSDSAVLLR |
| | | TRPVVIVEVSLNHPESVNRTNFDCVENGLPSVCMDLTLCFSYKG |
| | | KEVPGYIVLLYNMSLDVNRKIDSPSRFYFSSNGTSDVITGSMKV |
| | | SSKVPNCRTHQAFMRKDVRDILTPIQIEAAYRLGQHVIRKRSTE |
| | | EFPPLQPILQQKKERDIIEKTINFARFCAHENCSADLQVSARIG |
| | | FLKPHENKTYVAVGSMKTVMLNVSLFNAGDDAYETALHIRLPSG |
| | | LYFIKILDLEEKQINCEVTDSSGSVKLDCSIGYIYMDRLSRMDI |
| | | SFLLDVSSLSQAEEDLSLTVHATCANEREMDNLNKVTLAIPLKY |
| | | EVMLSVHGFVNPTSFIYGPKEENEPDTCMAEKMNFTFHVINTGH |
| | | SMAPNVSVEIMVPNSFAPQTDKLFNILDVQPAGECHFKTYQRKC |
| | | ALEQEKGAMKILKDIFTFLSKTDKKLLFCMKADPYCLTILCHLG |
| | | KMESGKEASVHIQLEGRPYLSEMDETSALKFEVRVTAFPEPNPK |
| | | VIELNKDENVAHVLLEGLHHQRPKRHFT |
| SEQ ID NO: 12 | α4 | VSASRPRPGSTPPPPPWQVYPVAEAWEGGASSSGSGEQGPRAGG |
| | | CGAPAGSSPKVLVAKSGARGLSSSWWGRRGDAQARGFGAGSWEL |
| | | EGDLAHVCAHLHGCPLGLWLVVGAPTARWLANASVVNPGAIYRC |
| | | RIGGNPGLTCEQLQLGSPSGEPCGKTCLEERDNQWLGVTLSRQP |
| | | GENGSIVTCGHRWKNIFYIKNENKLPMGVCYGMPSDLRTELSKR |
| | | IAPCYQDYVRKFGENFASCQAGISSFYTEDLIVMGAPGSSYWTG |
| | | SLFVYNITTNKYKAFLDRQNQVKFGSYLGYSVGAGHFRSPHTTE |
| | | VVGGAPQHEQIGKAYIFSIEAKELSILHEMKGKKLGSYFGASVC |
| | | AVDLNADGFSDLLVGAPMQSTIREEGRVFVYINSGSGAVMNEME |
| | | TELIGSDKYAARFGESIVNLGDIDNDGFEDVAVGAPQEDDLRGA |
| | | VYIYNGRADGISTAFSQRIEGFQISKSLSMFGQSISGQIDADNN |
| | | GYVDVAVGAFRSDSAVLLRTRPVVIVEVSLNHPESVNRTNFDCV |
| | | ENGLPSVCMDLTLCFSYKGKEVPGYIVLLYNMSLDVNRKIDSPS |
| | | RFYFSSNGTSDVITGSMKVSSKVPNCRTHQAFMRKDVRDILTPI |
| | | QIEAAYRLGQHVIRKRSTEEFPPLQPILQQKKERDIIEKTINFA |
| | | RFCAHENCSADLQVSARIGFLKPHENKTYVAVGSMKTVMLNVSL |
| | | FNAGDDAYETALHIRLPSGLYFIKILDLEEKQINCEVTDSSGSV |
| | | KLDCSIGYIYMDRLSRMDISFLLDVSSLSQAEEDLSLTVHATCA |
| | | NEREMDNLNKVTLAIPLKYEVMLSVHGFVNPTSFIYGPKEENEP |
| | | DTCMAEKMNFTFHVINTGHSMAPNVSVEIMVPNSFAPQTDKLFN |
| | | ILDVQPAGECHFKTYQRKCALEQEKGAMKILKDIFTFLSKTDKK |
| | | LLFCMKADPYCLTILCHLGKMESGKEASVHIQLEGRPYLSEMDE |
| | | TSALKFEVRVTAFPEPNPKVIELNKDENVAHVLLEGLHHQRPKR |
| | | HFT |
| SEQ ID NO: 13 | β7 | ELDAKISSAEKATEWRDPDLSLLGSCQPAPSCRECILSHPSCAW |
| | | CKQLFWGLGIRDQDASPFGSWGGPSPWPAHRCRPALWCLFCDPP |
| | | PPPPASAPRLSPGPSRRCTLDPLLCRRLHRAPCALCPAPCTLHP |
| | | ALRLGTPCATSTWPARPLAQPSPCPLPGFGSFVDKTVLPFVSTV |
| | | PAKLRHPCPTRLERCQPPRSFRHVLSLTGDATAFEREVGRQSVS |
| | | GNLDSPEGGFDAILQAALCQEKIGWRNVSRLLVFTSDDTFHTAG |
| | | DGKLGGIFMPSDGHCHLDSNGLYSRSPEFDYPSVGQVAQALSTA |
| | | NIQPIFAVTSATLPVYQELSKLIPKSAVGELSEDSSNVVQLIMD |
| | | AYNSLSSTVTLEHSALPPGVHISYESLCGDPEKREAEAGDRGQC |
| | | SHVPINHTVNFLVTLQATRCLPEPHLLRLRALGFSEELTVELHL |
| SEQ ID NO: 14 | β7 | ELDAKISSAEKATEQRDPDLSLLGSCQPAPSCRECILSHPSCAW |
| | | CKQLFWGLGIRDQDASPFGSWGGPSPWPAHRCRPALWCLFCDPP |
| | | PPPPASAPRLSPGPSRRCTLDPLLCRRLHRAPCALCPAPCTLHP |
| | | ALRLGTPCATSTWPARPLAQPSPCPLPGFGSFVDKTVLPFVSTV |
| | | PAKLRHPCPTRLERCQPPFSFRHVLSLTGDATAFEREVGRQSVS |
| | | GNLDSPEGGFDAILQAALCQEKIGWRNVSRLLVFTSDDTFHTAG |
| | | DGKLGGIFMPSDGHCHLDSNGLYSRSPEFDYPSVGQVAQALSTA |
| | | NIQPIFAVTSATLPVYQELSKLIPKSAVGELSEDSSNVVQLIMD |
| | | AYNSLSSTVTLEHSALPPGVHISYESLCGDPEKREAEAGDRGQC |
| | | SHVPINHTVNFLVTLQATRCLPEPHLLRLRALGFSEELTVELHT |
| | | LCDCNCSDTQPQAPHCSDGQGLLQCGVCSCAPGRLGRLCECSEA |
| | | ELSSPDLESGCRAPNGTGPLCSGKGRCQCGRCSCSGQSSGPLCE |
| | | CDDASCERHEGILCGGFGHCQCGRCHCHANRTGSACECSMDTDS |
| | | CLGPEGEVCSGHGDCKCNRCQCRDGYFGALCEQCSGCKTSCERH |
| | | RDCAECGAFGTGPLATNCSVACAHYNVTLALVPVLDDGWCKERT |
| | | LDNQLLFFLVEEEAGGMVVLTVRPQERGADH |
| SEQ ID NO: 15 | TAG | MGSSHHHHHHGSGLVPRGSASMSDSEVNQEAKPEVKPEVKPETH |
| | | INLKVSDGSSEIFFKIKKTTPLRRLMEAFAKRQGKEMDSLRFLY |
| | | DGIRIQADQTPEDLDMEDNDIIEAHREQIGG |
| SEQ ID NO: 16 | Flagellin | MGSSHHHHHHGSGLVPRGSASMSDSEVNQEAKPEVKPEVKPETH |
| | | INLKVSDGSSEIFFKIKKTTPLRRLMEAFAKRQGKEMDSLRFLY |
| | | DGIRIQADQTPEDLDMEDNDIIEAHREQIGGALTVNTNIASVTT |
| | | QVNLNKASTAQTTSMQRLSSGLRINSAKDDAAGLQIANRLTSQI |
| | | NGLGQAVKNANDGISIAQTEAGAMQASTDILQKMRTLALSSATG |
| | | SLSPDDRKSNNDEYQALTAELNRISATTTFGGQKLLDGSYGTKA |
| | | IQVGANANETINLTLDNVSAKSIGSQQLKTGNISISKDGLAAGE |
| | | LAVTGNGQTKTVNYGPGASAKDVAAQLNGAIGGLTATASTEVKL |
| | | DASGATAAAPANFDLTVGGSTVSFVGVTDNASLADQLKSNAAKL |
| | | GISVNYDESTKNLEIKSDTGENITFAPKAGAPGVKIAAKNGSGT |
| | | YGAAVPLNAAAGDKSVVTGQISLDSAKGYSIADGAGANGAGSTA |
| | | ALYGTGVTSVSSKKTNVSDTDVTSATNAQNAVAVIDKAIGSIDS |
| | | VRSGLGATQNRLTTTVDNLQNIQKNSTAARSTVQDVDFASETAE |
| | | LTKQQTLQQASTAILSQANQLPSSVLKLLQ |
| SEQ ID NO: 17 | OMPC | MGSSHHHHHHGSGLVPRGSASMSDSEVNQEAKPEVKPEVKPETH |
| | | INLKVSDGSSEIFFKIKKTTPLRRLMEAFAKRQGKEMDSLRFLY |
| | | DGIRIQADQTPEDLDMEDNDIIEAHREQIGGAEVYNKDGNKLDL |
| | | YGKVDGLHYFSDNKSEDGDQTYVRLGFKGETQVTDQLTGYGQWE |
| | | YQIQGNTSEDNKENSWTRVAFAGLKFQDVGSFDYGRNYGVVYDV |
| | | TSWTDVLPEFGGDTYGSDNFMQQRGNGFATYRNTDFFGLVDGLN |
| | | FAVQYQGKNGSVSGEGMTNNGRGALRQNGDGVGGSITYDYEGFG |
| | | IGAAVSSSKRTDDQNGSYTSNGVVRNYIGTGDRAETYTGGLKYD |
| | | ANNIYLAAQYTQTYNATRVGSLGWANKAQNFEAVAQYQFDFGLR |
| | | PSLAYLQSKGKNLGVINGRNYDDEDILKYVDVGATYYFNKNMST |
| | | YVDYKINLLDDNQFTRDAGINTDNIVALGLVYQF |
| SEQ ID NO: 18 | Poly-His tag | MGSSHHHHHHG |
| SEQ ID NO: 19 | Human S100-A8 | MLTELEKALNSIIDVYHKYSLIKGNFHAVYRDDLKKLLETECPQ |
| | | YIRKKGADVWFKELDINTDGAVNFQEFLILVIKMGVAAHKKSHE |
| | | ESHKE |
| SEQ ID NO: 20 | Human S100-A9 | MTCKMSQLERNIETIINTFHQYSVKLGHPDTLNQGEFKELVRKD |
| | | LQNFLKKENKNEKVIEHIMEDLDTNADKQLSFEEEFIMLMARLT |
| | | WASHEKMHEGDEGPGHHHKPGLGEGTP |
| SEQ ID NO: 21 | Human integrin | MAWEARREPGPRRAAVRETVMLLLCLGVPTGRPYNVDTESALLY |
| | α4 subunit | QGPHNTLFGYSVVLHSHGANRWLLVGAPTANWLANASVINPGAI |
| | | YRCRIGKNPGQTCELQLQLGSPNGEPCGKTCLEERDNQWLGVTL |
| | | SRQPGENGSIVTCGHRWKNIFYIKNENKLPTGGCYGVPPDLRTE |
| | | LSKRIAPCYQDYVKKFGENFASCQAGISSFYTKDLIVMGAPGSS |
| | | YWTGSLFVYNITTNKYKAFLDKQNQVKFGSYLGYSVGAGHFRSQ |
| | | HTTEVVGGAPQHEQIGKAYIFSIDEKELNILHEMKGKKLGSYFG |
| | | ASVCAVDLNADGFSDLLVGAPMQSTIREEGRVFVYINSGSGAVM |
| | | NAMETNLVGSDKYAARFGESIVNLGDIDNDGFEDVAIGAPQEDD |
| | | LQGAIYIYNGRADGISSTFSQRIEGLQISKSLSMFGQSISQIDA |
| | | DNNGYVDVAVGAFRSDSAVLLRTRPVVIVDASLSHPESVNRTKF |
| | | DCVENGWPSVCIDLTLCFSYKGKEVPGYIVLFYNMSLDVNRKAE |
| | | SPPRFYFSSNGTSDVITGSIQVSSREANCRTHQAFMRKDVRDIL |
| | | TPIQIEAAYHLGPHVISKRSTEEFPPLQPILQQKKEKDIMKKTI |
| | | NFARFCAHENCSADLQVSAKIGFLKPHENKTYLAVGSMKTLMLN |
| | | VSLFNAGDDAYETTLHVKLPVGLYFIKILELEEKQINCEVTDNS |
| | | GVVQLDCSIGYIYVDHLSRIDISFLLDVSSLSRAEEDLSITVHA |
| | | TCENEEEMDNLKHSRVTVAIPLKYEVKLTVHGFVNPTSFVYGSN |
| | | DENEPETCMVEKMNLTFHVINTGNSMAPNVSVEIMVPNSFSPQT |
| | | DKLFNILDVQTTTGECHFENYQRVCALEQQKSAMQTLKGIVRFL |
| | | SKTDKRLLYCIKADPHCLNFLCNFGKMESGKEASVHIQLEGRPS |
| | | ILEMDETSALKFEIRATGFPEPNPRVIELNKDENVAHVLLEGLH |
| | | HQRPKRYFTIVIISSSLLLGLIVLLLISYVMWKAGFFKRQYKSI |
| | | LQEENRRDSWSYINSKSNDD |
| SEQ ID NO: 21 | Human integrin | MNLQPIFWIGLISSVCCVFAQTDENRCLKANAKSCGECIQAGPN |
| | β1 subunit | CGWCTNSTFLQEGMPTSARCDDLEALKKKGCPPDDIENPRGSKD |
| | | IKKNKNVTNRSKGTAEKLKPEDITQIQPQQLVLRLRSGEPQTFT |
| | | LKFKRAEDYPIDLYYLMDLSYSMKDDLENVKSLGTDLMNEMRRI |
| | | TSDFRIGFGSFVEKTVMPYISTTPAKLRNPCTSEQNCTSPFSYK |
| | | NVLSLTNKGEVFNELVFKQRISGNLDSPEGGFDAIMQVAVCGSL |
| | | IGWRNVTRLLVFSTDAGFHFAGDGKLGGIVLPNDGQCHLENNMY |
| | | TMSHYYDYPSIAHLVQKLSENNIQTIFAVTEEFQPVYKELKNLI |
| | | PKSAVGTLSANSSNVIQLIIDAYNSLSSEVILENGKLSEGVTIS |
| | | YKSYCKNGVNGTGENGRKCSNISIGDEVQFEISITSNKCPKKDS |
| | | DSFKIRPLGFTEEVEVILQYICECECQSEGIPESPKCHEGNGTF |
| | | ECGACRCNEGRVGRHCECSTDEVNSEDMDAYCRKENSSEICSNN |
| | | GECVCGQCVCRKRDNTNEIYSGKFCECDNFNCDRSNGLICGGNG |
| | | VCKCRVCECNPNYTGSACDCSLDTSTCEASNGQICNGRGICECG |
| | | VCKCTDPKFQGQTCEMCQTCLGVCAEHKECVQCRAFNKGEKKDT |
| | | CTQECSYFNITKVESRDKLPQPVQPDPVSHCKEKDVDDCWFYFT |
| | | YSVNGNNEVMVHVVENPECPTGPDIIPIVAGVVAGIVLIGLALL |
| | | LIWKLLMIIHDRREFAKFEKEKMNAKWDTGENPIYKSAVTTVVN |
| | | PKYEGK |
| SEQ ID NO: 23 | Human integrin | MVALPMVLVLLLVLSRGESELDAKIPSTGDATEWRNPHLSMLGS |
| | β7 subunit | CQPAPSCQKCILSHPSCAWCKQLNFTASGEAEARRCARREELLA |
| | | RGCPLEELEEPRGQQEVLQDQPLSQGARGEGATQLAPQRVRVTL |
| | | RPGEPQQLQVRFLRAEGYPVDLYYLMDLSYSMKDDLERVRQLGH |
| | | ALLVRLQEVTHSVRIGFGSFVDKTVLPFVSTVPSKLRHPCPTRL |
| | | ERCQSPFSFHHVLSLTGDAQAFEREVGRQSVSGNLDSPEGGFDA |
| | | ILQAALCQEQIGWRNVSRLLVFTSDDTFHTAGDGKLGGIFMPSD |
| | | GHCHLDSNGLYSRSTEFDYPSVGQVAQALSAANIQPIFAVTSAA |
| | | LPVYQELSKLIPKSAVGELSEDSSNVVQLIMDAYNSLSSTVTLE |
| | | HSSLPPGVHISYESQCEGPEKREGKAEDRGQCNHVRINQTVTFW |
| | | VSLQATHCLPEPHLLRLRALGFSEELIVELHTLCDCNCSDTQPQ |
| | | APHCSDGQGHLQCGVCSCAPGRLGRLCECSVAELSSPDLESGCR |
| | | APNGTGPLCSGKGHCQCGRCSCSGQSSGHLCECDDASCERHEGI |
| | | LCGGFGRCQCGVCHCHANRTGRACECSGDMDSCISPEGGLCSGH |
| | | GRCKCNRCQCLDGYYGALCDQCPGCKTPCERHRDCAECGAFRTG |
| | | PLATNCSTACAHTNVTLALAPILDDGWCKERTLDNQLFFFLVED |
| | | DARGTVVLRVRPQEKGADHTQAIVLGCVGGIVAVGLGLVLAYRL |
| | | SVEIYDRREYSRFEKEQQQLNWKQDSNPLYKSAITTTINPRFQE |
| | | ADSPTL |

What is claimed is:

1. A method for detecting the presence and/or level of one or more inflammation-associated autoantibodies in a sample obtained from a human patient, wherein the autoantibodies are selected from one or more of
i) autoantibodies to a calprotectin,
iii autoantibodies to an integrin,
iii) autoantibodies to a lactoferritin,
iv) autoantibodies to a C-reactive protein,
comprising
contacting one or more antigens with said sample, wherein the one or more antigens are specific for the autoantibody of interest, and wherein the one or more antigens are bound to a substrate or detectable label, and
detecting the binding of said one or more one or more autoantibodies associated with inflammation to the one or more antigens.

2. The method of claim 1, further comprising classifying said sample as an inflammation sample or non-inflammation sample, wherein the presence or level of the one or one or more autoantibodies associated with inflammation, separately or in combination, correlates with presence of inflammation.

3. The method of claim 1, wherein a labeled antibody that specifically binds human immunoglobulin is used to detect the binding of the one or more one or more endogenous antibodies associated with inflammation to the one or more antigens.

4. The method of claim 1 wherein the patient exhibits clinical symptoms of a gastroinflammatory condition.

5. The method of claim 1 wherein the sample is whole blood, serum or plasma.

6. The method of claim 1 wherein the presence, severity and/or type of inflammation in the patient is associated with antibody class switching from IgG to IgA, for example such that the proportion of one or more endogenous antibodies associated with inflammation is higher in healthy patients and lower in patients with inflammation.

7. The method of claim 1 wherein the one or more one or more inflammation-associated autoantibodies are IgA antibodies.

8. The method of claim 1, wherein the immunoassay to detect the presence or level of one or more one or more inflammation-associated autoantibodies is an enzyme-linked immunosorbent assay (ELISA), an immunohistochemical assay, or an immunofluorescence assay.

9. The method of claim 1, further comprising detecting the presence or level in the sample of one or more additional antibodies selected from endogenous antibodies to polymorphonuclear leukocytes (PMNs or granulocytes, including neutrophil granulocytes), endogenous antibodies to microbes found in the gut, endogenous antibodies to food antigens, and combinations thereof

10. The method of claim 9, wherein the one or more additional antibodies comprise one or more of
a) anti-PMN antibody selected from the group consisting of an anti-PMN antibody (APMNA), perinuclear anti-PMN antibody (pAPMNA), and combinations thereof;
b) anti-yeast antibody selected from the group consisting of anti-yeast immunoglobulin A (AYA-IgA), anti-yeast immunoglobulin G (AYA-IgG), anti-yeast immunoglobulin M (AYA-IgM) and combinations thereof;
c) antimicrobial antibody selected from the group consisting of an anti-outer membrane protein C (ACA) antibody, anti-flagellin antibody (AFA), and combinations thereof.

11. The method of claim 1, wherein the one or more inflammation-associated autoantibodies comprise an autoantibody to a calprotectin or an autoantibody to an integrin.

12. The method of claim 1, wherein the one or more antigens are bound to one or more substrates, wherein the substrates comprise one or more microwell plates, such that where detecting binding to different antigens is desired, the different antigens are on different microwell plates or in different wells of the same microwell plate.

13. The method of claim 1, wherein the one or more antigens are bound to one or more substrates, comprising the steps of
a) Affixing the one or more antigens to their respective substrates,
b) Blocking any uncoated surfaces of the substrates with protein,
c) Exposing the antigens to the sample to allow formation of antigen-antibody complexes,
d) Exposing the antigen-antibody complexes thus formed to a labeled antibody that binds immunoglobulin of the patient,
e) Detecting binding of the labeled antibody to the antigen-antibody complexes.

14. The method of claim 1 wherein the inflammation-associated autoantibody is autoantibody to calprotectin and the antigen is a calprotectin S100A8/S100A9 heterodimer bound to a substrate.

15. The method of claim 1 wherein the inflammation-associated autoantibody is autoantibody to integrin and the antigen is an integrin alpha-4/beta-7 heterodimer bound to a substrate.

16. A method of treating an inflammatory condition in a patient comprising detecting the presence and/or level of one or more one or more inflammation-associated autoantibodies in accordance with claim 1, and administering to said patient a therapeutically effective amount of a drug useful for treating one or more symptoms associated with the inflammatory condition.

17. The method of claim 16 wherein the inflammatory condition is inflammatory bowel disease (IBD).

18. The method of claim 17, wherein said drug is selected from the group known to physicians consisting of aminosalicylates, corticosteroids, thiopurines, methotrexate, monoclonal antibodies, free bases thereof, pharmaceutically acceptable salts thereof, derivatives thereof, analogs thereof, and combinations thereof.

19. A reagent comprising an isolated peptide selected from a human calprotectin or antigenic fragment thereof, a human β-integrin or antigenic fragment thereof, a human lactoferritin or antigenic fragment thereof, and a human C-reactive protein or antigenic fragment thereof, wherein the isolated peptide is bound to one or more of a label, a purification tag, a solid substrate, or another protein.

20. The reagent of claim 19 wherein the isolated peptide s a calprotectin or antigenic fragment thereof, comprising at least 10 consecutive amino acids in a sequence from a wild type human calprotectin, wherein the calprotectin or antigenic fragment thereof is bound to one or more of a label, a purification tag, a solid substrate, or another protein.

19. The reagent claim 19 wherein the isolated peptide is an integrin or antigenic fragment thereof, comprising at least 10 consecutive amino acids in a sequence from a wild type human integrin, wherein the integrin or antigenic fragment thereof is bound to one or more of a label, a purification tag, a solid substrate, or another protein.

22. The reagent of claim 19, wherein the isolated peptide is bound to a poly-histidine tag.

23. The reagent of claim 19, comprising
a) a fusion protein comprising a calprotectin S100A8 monomer region, with sequence comprising at least 20 amino acid residues in sequence from a human calprotectin S100A8 monomer, and a calprotectin S100A9 monomer region, with sequence comprising at least 20 amino acid residues in sequence from a human calprotectin S100A9 monomer, wherein the regions are linked by a linker sequence, optionally further comprising a polyhistidine sequence and one or more additional residues to enhance solubility; or
b) a fusion peptide comprising an integrin α (alpha) subunit region, comprising at least 20 amino acid residues in sequence from a human integrin α (alpha) subunit, and an integrin β (beta) subunit region, comprising at least 20 amino acid residues in sequence from a human integrin β (beta) subunit, wherein the regions are linked by a linker sequence, optionally further comprising a polyhistidine sequence and one or more additional residues to enhance solubility.

24. The reagent of claim 23, wherein the linker sequence is a sequences of 10-30 residues selected from glycine residues, serine residues, and combinations thereof.

25. A diagnostic kit comprising a reagent according to claim 19 and further comprising labeled antibody specific for immunoglobulin and capable of binding to a complex formed between the reagent and an inflammation-associated autoantibody.

26. A bacterial expression construct comprising
a) a promoter operably linked to
b) an open reading frame encoding a protein for use in or as reagent according to claim 19.

27. A bacterial cell line comprising the expression construct of claim 26.


SEQUENCE LISTING (23 sequences)

SEQ ID NO: 1 - protein, 243 aa, Artificial Sequence
  Hetero-chimeric S100A8/S100A9
MGSSHHHHHHGLTELESAINSLIEVYHKYSLVKGNYHALYRDDLKKLLETECPQYMKKKDADTWFQELDVNSDGAINFEEFLILVIKVGVASHKDIHKEGGGGSGGGGSGGGGSADQMSQLECSIETIINIFHQYSVRLEHPDKLNQKEMKQLVKKELPNFLKKQKKNDNAINKIMEDLDTNGDKELNFEEFSILVARLTVASHEEMHKNAPEGEGHSHGPGFGEGSQGHCHSHGGHGHGHSH

SEQ ID NO: 2 - protein, 102 aa, Artificial Sequence
  S100A12
MGSSHHHHHHGTKLEDHLEGIVDVFHRYSARVGHPDTLSKGEMKQLIIRELPNTLKNTKDQATVDKLFQDLDADKDGQVNFNEFISLVSVVLDTSHKNTHKE

SEQ ID NO: 3 - protein, 99 aa, Artificial Sequence
  S100A8
MGSSHHHHHHGLTELESAINSLIEVYHKYSLVKGNYHALYRDDLKKLLETECPQYMKKKDADTWFQELDVNSDGAINFEEFLILVIKVGVASHKDIHKE

SEQ ID NO: 4 - protein, 140 aa, Artificial Sequence
  S100A9
  misc_feature (130)..(130): Xaa can be any naturally occurring amino acid
MGSSHHHHHHGADQMSQLECSIETIINIFHQYSVRLEHPDKLNQKEMKQLVKKELPNFLKKQKKNDNAINKIMEDLDTNGDKELNFEEFSILVARLTVASHEEMHKNAPEGEGHSHGPGFGEGSQGFFIXHGGHGHGHSH

SEQ ID NO: 5 - DNA, 21 bp, Canis familiaris
gtgtctgcctctcgacctcgg

SEQ ID NO: 6 - DNA, 27 bp, Canis familiaris
cagagaattgaaggatttcaaatcagc

SEQ ID NO: 7 - DNA, 27 bp, Canis familiaris
ttatgtgaaatgacgtttgggtctttg

SEQ ID NO: 8 - DNA, 21 bp, Canis familiaris
gaattggatgccaagatctcc

SEQ ID NO: 9 - DNA, 27 bp, Canis familiaris
ttacagtgtgtgcagctccacagtcag

SEQ ID NO: 10 - DNA, 24 bp, Canis familiaris
ttagtgatccgcgcctctctcttg

SEQ ID NO: 11 - protein, 909 aa, Canis familiaris
WLVVGAPTARWLANASVVNPGAIYRCRIGGNPGLTCEQLQLGSPSGEPCGKTCLEERDNQWLGVTLSRQPGENGSIVTCGHRWKNIFYIKNENKLPMGVCYGMPSDLRTELSKRIAPCYQDYVRKFGENFASCQAGISSFYTEDLIVMGAPGSSYWTGSLFVYNITTNKYKAFLDRQNQVKFGSYLGYSVGAGHFRSPHTTEVVGGAPQHEQIGKAYIFSIEAKELSILHEMKGKKLGSYFGASVCAVDLNADGFSDLLVGAPMQSTIREEGRVFVYINSGSGAVMNEMETELIGSDKYAARFGESIVNLGDIDNDGFEDVAVGAPQEDDLRGAVYIYNGRADGISTAFSQRIEGFQISKSLSMFGQSISGQIDADNNGYVDVAVGAFRSDSAVLLRTRPVVIVEVSLNHPESVNRTNFDCVENGLPSVCMDLTLCFSYKGKEVPGYIVLLYNMSLDVNRKIDSPSRFYFSSNGTSDVITGSMKVSSKVPNCRTHQAFMRKDVRDILTPIQIEAAYRLGQHVIRKRSTEEFPPLQPILQQKKERDIIEKTINFARFCAHENCSADLQVSARIGFLKPHENKTYVAVGSMKTVMLNVSLFNAGDDAYETALHIRLPSGLYFIKILDLEEKQINCEVTDSSGSVKLDCSIGYIYMDRLSRMDISFLLDVSSLSQAEEDLSLTVHATCANEREMDNLNKVTLAIPLKYEVMLSVHGFVNPTSFIYGPKEENEPDTCMAEKMNFTFHVINTGHSMAPNVSVEIMVPNSFAPQTDKLFNILDVQPAGECHFKTYQRKCALEQEKGAMKILKDIFTFLSKTDKKLLFCMKADPYCLTILCHLGKMESGKEASVHIQLEGRPYLSEMDETSALKFEVRVTAFPEPNPKVIELNKDENVAHVLLEGLHHQRPKRHFT

SEQ ID NO: 12 - protein, 1015 aa, Canis familiaris
VSASRPRPGSTPPPPPWQVYPVAEAWEGGASSSGSGEQGPRAGGCGAPAGSSPKVLVAKSGARGLSSSWWGRRGDAQARGFGAGSWELEGDLAHVCAHLHGCPLGLWLVVGAPTARWLANASVVNPGAIYRCRIGGNPGLTCEQLQLGSPSGEPCGKTCLEERDNQWLGVTLSRQPGENGSIVTCGHRWKNIFYIKNENKLPMGVCYGMPSDLRTELSKRIAPCYQDYVRKFGENFASCQAGISSFYTEDLIVMGAPGSSYWTGSLFVYNITTNKYKAFLDRQNQVKFGSYLGYSVGAGHFRSPHTTEVVGGAPQHEQIGKAYIFSIEAKELSILHEMKGKKLGSYFGASVCAVDLNADGFSDLLVGAPMQSTIREEGRVFVYINSGSGAVMNEMETELIGSDKYAARFGESIVNLGDIDNDGFEDVAVGAPQEDDLRGAVYIYNGRADGISTAFSQRIEGFQISKSLSMFGQSISGQIDADNNGYVDVAVGAFRSDSAVLLRTRPVVIVEVSLNHPESVNRTNFDCVENGLPSVCMDLTLCFSYKGKEVPGYIVLLYNMSLDVNRKIDSPSRFYFSSNGTSDVITGSMKVSSKVPNCRTHQAFMRKDVRDILTPIQIEAAYRLGQHVIRKRSTEEFPPLQPILQQKKERDIIEKTINFARFCAHENCSADLQVSARIGFLKPHENKTYVAVGSMKTVMLNVSLFNAGDDAYETALHIRLPSGLYFIKILDLEEKQINCEVTDSSGSVKLDCSIGYIYMDRLSRMDISFLLDVSSLSQAEEDLSLTVHATCANEREMDNLNKVTLAIPLKYEVMLSVHGFVNPTSFIYGPKEENEPDTCMAEKMNFTFHVINTGHSMAPNVSVEIMVPNSFAPQTDKLFNILDVQPAGECHFKTYQRKCALEQEKGAMKILKDIFTFLSKTDKKLLFCMKADPYCLTILCHLGKMESGKEASVHIQLEGRPYLSEMDETSALKFEVRVTAFPEPNPKVIELNKDENVAHVLLEGLHHQRPKRHFT

SEQ ID NO: 13 - protein, 440 aa, Canis familiaris
ELDAKISSAEKATEWRDPDLSLLGSCQPAPSCRECILSHPSCAWCKQLFWGLGIRDQDASPFGSWGGPSPWPAHRCRPALWCLFCDPPPPPPASAPRLSPGPSRRCTLDPLLCRRLHRAPCALCPAPCTLHPALRLGTPCATSTWPARPLAQPSPCPLPGFGSFVDKTVLPFVSTVPAKLRHPCPTRLERCQPPFSFRHVLSLTGDATAFEREVGRQSVSGNLDSPEGGFDAILQAALCQEKIGWRNVSRLLVFTSDDTFHTAGDGKLGGIFMPSDGHCHLDSNGLYSRSPEFDYPSVGQVAQALSTANIQPIFAVTSATLPVYQELSKLIPKSAVGELSEDSSNVVQLIMDAYNSLSSTVTLEHSALPPGVHISYESLCGDPEKREAEAGDRGQCSHVPINHTVNFLVTLQATRCLPEPHLLRLRALGFSEELTVELHL

SEQ ID NO: 14 - protein, 691 aa, Canis familiaris
ELDAKISSAEKATEWRDPDLSLLGSCQPAPSCRECILSHPSCAWCKQLFWGLGIRDQDASPFGSWGGPSPWPAHRCRPALWCLFCDPPPPPPASAPRLSPGPSRRCTLDPLLCRRLHRAPCALCPAPCTLHPALRLGTPCATSTWPARPLAQPSPCPLPGFGSFVDKTVLPFVSTVPAKLRHPCPTRLERCQPPFSFRHVLSLTGDATAFEREVGRQSVSGNLDSPEGGFDAILQAALCQEKIGWRNVSRLLVFTSDDTFHTAGDGKLGGIFMPSDGHCHLDSNGLYSRSPEFDYPSVGQVAQALSTANIQPIFAVTSATLPVYQELSKLIPKSAVGELSEDSSNVVQLIMDAYNSLSSTVTLEHSALPPGVHISYESLCGDPEKREAEAGDRGQCSHVPINHTVNFLVTLQATRCLPEPHLLRLRALGFSEELTVELHTLCDCNCSDTQPQAPHCSDGQGLLQCGVCSCAPGRLGRLCECSEAELSSPDLESGCRAPNGTGPLCSGKGRCQCGRCSCSGQSSGPLCECDDASCERHEGILCGGFGHCQCGRCHCHANRTGSACECSMDTDSCLGPEGEVCSGHGDCKCNRCQCRDGYFGALCEQCSGCKTSCERHRDCAECGAFGTGPLATNCSVACAHYNVTLALVPVLDDGWCKERTLDNQLLFFLVEEEAGGMVVLTVRPQERGADH

SEQ ID NO: 15 - protein, 119 aa, Artificial Sequence
  TAG
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 16 - protein, 602 aa, Artificial Sequence
  Flagellin
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGALTVNTNIASVTTQVNLNKASTAQTTSMQRLSSGLRINSAKDDAAGLQIANRLTSQINGLGQAVKNANDGISIAQTAEGAMQASTDILQKMRTLALSSATGSLSPDDRKSNNDEYQALTAELNRISATTTFGGQKLLDGSYGTKAIQVGANANETINLTLDNVSAKSIGSQQLKTGNISISKDGLAAGELAVTGNGQTKTVNYGPGASAKDVAAQLNGAIGGLTATASTEVKLDASGATAAAPANFDLTVGGSTVSFVGVTDNASLADQLKSNAAKLGISVNYDESTKNLEIKSDTGENITFAPKAGAPGVKIAAKNGSGTYGAAVPLNAAAGDKSVVTGQISLDSAKGYSIADGAGANGAGSTAALYGTGVTSVSSKKTNVSDTDVTSATNAQNAVAVIDKAIGSIDSVRSGLGATQNRLTTTVDNLQNIQKNSTAARSTVQDVDFASETAELTKQQTLQQASTAILSQANQLPSSVLKLLQ

SEQ ID NO: 17 - protein, 474 aa, Artificial Sequence
  OMPC
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEVYNKDGNKLDLYGKVDGLHYFSDNKSEDGDQTYVRLGFKGETQVTDQLTGYGQWEYQIQGNTSEDNKENSWTRVAFAGLKFQDVGSFDYGRNYGVVYDVTSWTDVLPEFGGDTYGSDNFMQQRGNGFATYRNTDFFGLVDGLNFAVQYQGKNGSVSGEGMTNNGRGALRQNGDGVGGSITYDYEGFGIGAAVSSSKRTDDQNGSYTSNGVVRNYIGTGDRAETYTGGLKYDANNIYLAAQYTQTYNATRVGSLGWANKAQNFEAVAQYQFDFGLRPSLAYLQSKGKNLGVINGRNYDDEDILKYVDVGATYYFNKNMSTYVDYKINLLDDNQFTRDAGINTDNIVALGLVYQF

SEQ ID NO: 18 - protein, 11 aa, Artificial Sequence
  Poly-His tag
MGSSHHHHHHG

SEQ ID NO: 19 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 20 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 21 - protein, 1032 aa, Homo sapiens [sequence omitted]

SEQ ID NO: 22 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 23 - protein, 798 aa, Homo sapiens
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL